(19) United States
(12) Patent Application Publication
(10) Pub. No.: US 2010/0330085 A1
(43) Pub. Date: Dec. 30, 2010

(54) METHOD OF TREATMENT, PROPHYLAXIS AND DIAGNOSIS OF PATHOLOGIES OF THE BONE

(75) Inventors: ANNA KATHLEEN COUSSENS, CLEARVIEW (AU); ANGELA MARY VAN DAAL, MERMAID BEACH (AU); BARRY CRAMPTON POWELL, ERINDALE (AU); PETER JOHN ANDERSON, CLARENCE PARK (AU)

Correspondence Address: KNOBBE MARTENS OLSON & BEAR LLP, 2040 MAIN STREET, FOURTEENTH FLOOR, IRVINE, CA, 92614, US

(73) Assignee: WOMEN'S & CHILDREN'S HEALTH RESEARCH INSTITUTE INC., North Adelaide (AU)

(21) Appl. No.: 12/841,697

(22) Filed: Jul. 22, 2010

Related U.S. Application Data
(63) Continuation of application No. 12/161,686, filed on Jul. 21, 2008, now abandoned.

(30) Foreign Application Priority Data
Jan. 20, 2006 (AU) 2006900307

(51) Int. Cl. A61K 39/395; A61K 31/7088; A61K 31/713; A61K 38/00; C07H 21/04; C07H 21/02; C07K 16/00; C07K 2/00; A61P 19/00; A61P 35/00; C40B 30/00

(52) U.S. Cl. 424/133.1; 514/44 R; 514/44 A; 514/16.7; 424/172.1; 536/23.1; 536/24.5; 530/387.1; 530/387.3; 530/300; 506/7

(57) ABSTRACT

The present invention relates generally to the fields of treatment, prophylaxis and diagnosis. More particularly, the present invention identifies genes and gene products associated with bone morphogenesis and pathologies of the bone. Even more particularly, the present invention contemplates the regulation of expression of these genes or the activity of the gene products in the treatment, prophylaxis and diagnosis of bone pathologies. Cell-based therapies and manipulation of cells in in vitro culture also form part of the present invention.

### RELATED APPLICATIONS

This application is a continuation of U.S. application Ser. No. 12/161,686, filed Jul. 21, 2008, which is the U.S. National Phase of International Application PCT/AU2007/000055, filed Jan. 19, 2007 designating the U.S., and published in English as WO 2007/082352 on Jul. 26, 2007, which claims priority to Australian Patent Application No. 2006900307, filed Jan. 20, 2006.

### FIELD OF INVENTION

The present invention relates generally to the fields of treatment, prophylaxis and diagnosis. More particularly, the present invention identifies genes and gene products associated with bone morphogenesis and pathologies of the bone. Even more particularly, the present invention contemplates the regulation of expression of these genes or the activity of the gene products in the treatment, prophylaxis and diagnosis of bone pathologies. Cell-based therapies and manipulation of cells in in vitro culture also form part of the present invention.

### DESCRIPTION OF THE PRIOR ART

Reference to any prior art in this specification is not, and should not be taken as, an acknowledgment or any form of suggestion that that prior art forms part of the common general knowledge in any country.

Bibliographic details of references cited herein are collected at the end of the subject specification.

Bone pathologies including bone cancers, fractures, craniosynostosis, osteoporosis and other biochemical or structural deficiencies can cause severe impairment, loss of quality of life and premature death to affected subjects. Surgical or other physical intervention has been the major method of dealing with many of these pathologies. There is a need to be able to treat or prevent or assist in the repair of bone-associated disorders by medicinal intervention.

At birth, the human skull is comprised of 45 bony elements, separated by fibrous joints known as sutures (Wilkie and Morriss-Kay, Nat Rev Genet. 2(6):458-468, 2001). The bones which surround the brain are called calvaria and develop through intramembranous ossification. This is in contrast to the bones which comprise the cranial base and facial region, which form by the more common method of endochondral ossification. For normal skull development the calvarial sutures need to remain as fibrous joints (unfused) until the brain has stopped growing and the rest of the skull, particularly the facial region, stops growing to allow for the movement of bones in relation to the new growth.

Calvarial bones first form from the condensation of ectomesenchyme (primary center of ossification) and then differentiation into osteoprogenitors, preosteoblasts and finally osteoblasts which secrete a collagen-proteoglycan extracellular matrix (ECM). Mineralization of the ECM traps osteoblasts which differentiate into osteocytes. The signals that initiate the bone formation are unclear. Once the primary ossification center is set up, growing bone radiates out from the primary center of ossification forming flat bony spicules. At approximately 18 weeks gestation, the growing osteogenic fronts meet and sutures are formed at these margins (Dixon et al, Fundamentals of Craniofacial Growth, New York, CRC Press, 1997).

Normal suture fusion does not occur until after birth and, with the exception of the metopic suture, may not occur until adulthood (Cohen Am J Med Genet. 47(5):581-616, 1993). The growth of the skull, however, ceases by the age of 4-6 years with most growth occurring during the first 6 months of life (Enlow, Facial Growth, Philadelphia, WB Saunders Co., 1990). The brain ceases growth before the calvarial sutures stop growing and finally fuse, so it seems the growth of the brain is not entirely responsible for cranial vault growth (Cohen 1993 supra). Cessation of growth does not always lead to fusion (Dixon et al, 1997 supra). The cause of suture fusion is still unclear and may involve many factors, including hormonal, genetic, mechanical and local factors (Persson et al, Journal of Anatomy 125:313-321, 1978).

Fusion starts with the cessation of proliferation and the differentiation of preosteoblast into osteoblasts at the osteogenic fronts. The margins of the sutures gradually encroach into the intervening space, changing from a flat edge to interdigitation. Final differentiation results in the replacement of simple trabeculae bone with a multi-laminated network (Cohen, 1993 supra). Apoptosis also appears to play a role in the maintenance of sutures. If sutures get too close during growth, then apoptosis of adjoining cells occurs, keeping the bones separated. Deregulation of apoptosis could therefore lead to early fusion (Furtwangler et al, Acta Anat 124(1-2):74-80, 1985).

Premature fusion of calvarial sutures, known as craniosynostosis, occurs in 1 in 2500 live births in the Western World (Wilkie, Am J Med Genet. 90(1):82-84, 2000) and is the second most common cranial defect. Fusion can be due to either premature bony bridging between apposed bones, or increased bone growth resulting in extremely overlapped bones. It can occur at only one or multiple calvarial sutures, it may occur before or after birth and it may be sporadic or syndromic. The study of craniosynostosis is important because it provides a model to study the causes of suture fusion bone growth and differentiation and, therefore, the factors regulating sutural maintenance and fusion. The known causes of craniosynostosis are varied, including monogenic conditions due to gene mutations, metabolic disorders, haematologic disorders and teratogens.

At least 5 genes have been identified to cause syndromic craniosynostosis, however the sporadic forms are not as well understood. There are over 100 syndromes that show some form of craniosynostosis. The most common dominantly inherited forms are Apert, Beare-Stevenson, Boston, Crouzon, Jackson-Weiss, Pfeiffer, Saethre-Chotzen and Muenke syndrome (reviewed by Muenke and Wilkie Craniosynostosis Syndromes 3:6117-6146, 2000). The phenotypes of these syndromes are not consistent between patients even with the same mutations. However, all are associated with multiple facial and limb abnormalities.

Invasive surgery is required to correct the facial abnormalities caused by craniosynostosis, by re-opening the fused section of tissue. In non-syndromic cases, removal of the fused tissue allows the skull to regrow normally and bone is replaced within a short period of time. In most syndromic cases, however, multiple surgeries are required to continually re-open the tissues as the bones re-fuse too early. The identification of therapeutic agents which would limit the premature re-fusion of the sutures, or which can be used to stop premature fusion of the sutures, especially when the presence of a genetic abnormality is first detected, would be highly desirable.

The culturing of suture mesenchyme is a common method to study the process of suture mesenchyme differentiation in vitro. There is a need, therefore, to identify biomarkers which assist in determining the stage of differentiation of osteoblasts. The ECM of calvarial sutures consists of 90% type 1 collagens (α1(I) coll and α2(I) coll), cell adhesion proteins (osteopontin (OP), fibronectin and thrombospondin), calcium-binding proteins (osteonectin (ON), and bone sialoprotein (BSP)), proteins involved in mineralization (osteocalcin (OC)) and enzymes (collagenase and alkaline phosphatase (ALP)) [Ducy et al, Cell 89(5):747-754, 1997; Robbins, Robbins Pathologic Basis of Disease, 1999]. Collagen is the earliest factor expressed in osteoprogenitor cells, followed by ALP, ON, BSP and finally OC in post-proliferative osteoblasts. OP is expressed virtually in all proliferating osteoprogenitor and preosteoblast cells, while low BSP expression has also been identified before collagen expression in progenitor cells (Liu et al, J Cell Sci 116(9):1787-1796, 2003). The current markers of osteoblast phenotype are, therefore, Col1, ALP, BSP, ON and OC. Research shows, however, that these markers have a large overlap of expression and do not uniquely identify the cells from each stage of differentiation (Liu et al, 2003 supra).

A study of the differences in gene expression between unfused suture mesenchyme and fused calvarial suture bone would identify genes involved in osteogenesis. These genes would both promote cellular proliferation and maintenance of the early osteoblasts populations (mesenchyme specific) in addition to those which promote osteoblast differentiation and bone mineralization (fused suture specific).

### SUMMARY

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

All scientific citations, patents, patent applications and manufacturer's technical specifications referred to hereinafter are incorporated herein by reference in their entirety.

In accordance with the present invention, differentially expressed genes associated with bone pathologies have been identified. The identification of these genes enables the development of therapeutic, prophylactic, diagnostic and tissue culture protocols in the treatment, prophylaxis and management of a range of bone pathologies including bone cancer, bone resorption and repair, bone fracture, suture-based cranial abnormalities including craniosynostosis, cytoskeletal disorders, osteoporosis, mineralization deficiencies and other biochemical or structural deficiencies. The present invention further enables the promotion of bone health including bone growth. The genes may also be considered as biomarkers for bone pathologies and hence may be useful in the diagnosis of a range of disorders or risk of development of same or a state of bone health. Manipulation of gene expression or the activity of the gene product is also useful in in vitro cell culture protocols such as in the development of adipose-derived stromal cells, bone marrow-derived stromal cells, osteoclasts and osteoblasts.

Without wishing to limit the present invention to any one theory or mode of action it is proposed that the genes identified relate generally to bone cell proliferation (as typified by unfused sutures) and bone cell differentiation (as typified by fusing sutures).

Accordingly, one aspect of the present invention contemplates a method for the treatment or prophylaxis of a bone pathology including reducing the risk of developing a bone pathology in a subject, said method comprising administering to said subject an effective amount of an agent which modulates expression of genetic material or the activity of encoded products of the genetic material wherein the genetic material is differentially expressed in unfused versus fused calvarial sutures.

The present invention further provides the use of a set of biomarkers comprising one or more genes or gene products differentially expressed in unfused sutures compared to fused calvarial sutures in a subject in the manufacture of a medicament or development of a diagnostic protocol for a bone pathology.

The preferred subject is a human. Reference to a “biomarker” includes a gene or gene product. A “gene product” includes a protein or RNA.

A list of abbreviations used in the subject specification is provided in Table 1.

TABLE 1
List of Abbreviations

| ABBREVIATION | DESCRIPTION |
|---|---|
| α1(I) coll | α1 Type I collagen |
| α2(I) coll | α2 Type I collagen |
| ALP | Alkaline phosphatase |
| BSP | Bone sialoprotein |
| ECM | Extracellular matrix |
| OC | Osteocalcin |
| ON | Osteonectin |
| OP | Osteopontin |

### BRIEF DESCRIPTION OF THE FIGURES

Some figures contain color representations or entities. Color photographs are available from the Patentee upon request or from an appropriate Patent Office. A fee may be imposed if obtained from a Patent Office.

FIG. 1 is a graphical representation of expression levels of the top 8 differentially regulated genes between fused and unfused sutures.

FIGS. 2(a) and (c) and FIG. 2(b) are graphical and photographic representations, respectively, which show mRNA and protein validation of differential expression identified by microarray analysis. (a) Real-time QRT-PCR analysis of six genes with increased expression in unfused sutures (C1QTNF3 short isoform, RBP4, GPC3, PTN, PRELP, FMOD) and three genes with increased expression in fused sutures (ANXA3, WIF1 and CYFIP2) for unfused, fusing and fused suture tissue isolated from sagittal, coronal, lambdoid and metopic sutures. Mean expression+SEM is shown (b) Western blot analysis of individual protein samples in the order seen in (c), for collagen type 1 (COL1), GPC3, C1QTNF3 and RBP4. (c) Densitometry analysis of western blots normalized to COL1 expression. Numbers in (b) and (c) refer to patient numbers.

FIGS. 3(a) through (c) are photographic representations showing the expression pattern of two of the preferred genes in sutures. (a-b) Immunofluorescence and H&E stain showing localization (yellow) of RBP4 in the cytoplasm of osteocytes (oc) in ectocranial surface bone (unfused coronal suture) (c-d) Serial immunofluorescence (c) and H&E sections (d) showing RBP4 located in cells in the region between calcified tissue (bn) and mesenchyme (m) (unfused left lambdoid suture). (e) RBP4 was not detected on the endocranial surface of unfused sutures (coronal). (f) RBP4 was localized to the cytoplasm of osteoblasts (ob) lining the developing bone, those being trapped in the osteoid (arrow head), and osteocytes (unfused coronal suture). (g) Corresponding phase contrast image to the central region in (f). (h) RBP4 was not detected in fused sutures. Red blood cells had weak autofluorescence (sagittal). (i-l) GPC3 immunofluorescence and H&E detected protein in mesenchymal cells close to the tissue surface (arrow head) in the mid-suture region (i). Membrane staining was observed for the cytoplasmic extensions of mesenchymal cells adjacent to calcified bone (k-l). (j) H&E of section deep to (k) showing calcified bone protruding into intervening mesenchyme with osteoblasts lining the bone. Scale: 10 μm.

### DETAILED DESCRIPTION OF THE INVENTION

The present invention provides differentially expressed genes in the form of biomarkers which define targets for therapeutic, prophylactic and diagnostic protocols for bone pathologies in a subject. The biomarkers comprise a set of genes or gene products differentially expressed in unfused sutures compared to fused sutures. In particular, the unfused sutures versus fused sutures are in a subject with a suture-based cranial abnormality, such as, but not limited to, craniosynostosis. However, the identification of the biomarkers enables therapeutic, prophylactic and diagnostic protocols to be developed for a range of bone pathologies including bone cancers, bone resorption and repair, fracture management, suture-based cranial abnormalities such as craniosynostosis, cytoskeletal disorders, osteoporosis, and mineralization deficiencies or other biochemical or structural deficiencies. The biomarkers are also useful in promoting bone growth or maintaining a state of bone health. The biomarkers are referred to herein as a “target gene” or “target protein” or “target gene expression product”.

In describing and claiming the present invention, the following terminology is used in accordance with the definitions set forth below.

It is to be understood that unless otherwise indicated, the subject invention is not limited to specific genes, assay techniques, physiological conditions or the like, as such may vary. It is also to be understood that the terminology used herein is for the purpose of describing particular embodiments only and is not intended to be limiting.

The singular forms “a”, “an” and “the” include plural aspects unless the context clearly dictates otherwise. Thus, for example, reference to “a gene” includes a single gene, as well as two or more genes; reference to “an agent” includes reference to a single agent or two or more agents; reference to “the bone pathology” includes one bone pathology or multiple bone pathologies; and so on.

The term “bone pathology” includes any disorder or deficiency in the bone including but not limited to conditions of bone cancer, deficient bone mineralization or where bone repair is required such as following a fracture, green stick or bone chip, suture-based cranial disorders such as craniosynostosis, cytoskeletal disorders, osteoporosis or other biochemical or structural deficiencies. The term “bone pathology” is not to be considered limiting to any one condition, disease or deficiency. One particular condition, however, is craniosynostosis. The term “bone pathology” also refers to a level of bone health. Hence, certain target genes or gene products may be useful in maintaining or promoting bone health.

A wide variety of conditions that result in loss of bone mineral content, for example, is contemplated by the present invention. Subjects with such conditions may be identified through clinical diagnosis utilizing well known techniques. Representative examples of diseases that may be treated included dysplasias, wherein there is abnormal growth or development of bone such as in achondroplasia, cleidocranial dysostosis, enchondromatosis, fibrous dysplasia, Gaucher's disease, hypophosphatemic rickets, Marfan's syndrome, multiple hereditary exostoses, neurofibromatosis, osteogenesis imperfecta, oesteopetrosis, osteopoikilosis, sclerotic lesions, fractures, periodontal disease, pseudoarthrosis and pyogenic osteomyelitis.

Another condition contemplated herein includes bone cancer wherein there is abnormal growth of bone cells in bone or other tissue to which bone cells have metastasized.

Other conditions contemplated herein include a wide variety of causes of osteopenia (i.e. a condition that causes greater than one standard deviation of bone mineral content or density below peak skeletal mineral content at youth). Representative examples of such conditions include those conditions caused by anemia, steroids, heparin, scurvy, malnutrition, calcium deficiency, idiopathic osteoporosis, congenital osteopenia or osteoporosis, transient regional osteoporosis and osteomalacia.

The term “craniosynostosis” refers to the premature fusion of calvarial sutures. The condition may arise from any number of conditions including Apert, Beare-Stevenson, Boston, Crouzon, Jackson-Weiss, Pfeiffer, Saethre-Chotzen and Muenke syndrome. Over 100 syndromes can cause craniosynostosis (see Muenke and Wilkie, 2000 supra).

As indicated above, there may be situations when it is important to assist bone growth or to facilitate bone health maintenance. The term “pathology”, therefore, does not necessarily mean the treatment of a disease condition. Situations where the subject biomarkers may be useful for non-disease conditions is in the elderly, young infants, athletes and non-human animals such as horses. Furthermore, bone growth may be promoted in subjects where it is sub-optimal; bone growth may be inhibited in subjects with excessive bone growth; and bone cancer growth can be inhibited.

The terms “compound”, “agent”, “chemical agent”, “pharmacologically active agent”, “medicament”, “active” and “drug” are used interchangeably herein to refer to a chemical compound that induces a desired pharmacological and/or physiological effect. The terms also encompass pharmaceutically acceptable and pharmacologically active ingredients of those active agents specifically mentioned herein including but not limited to salts, esters, amides, prodrugs, active metabolites, analogs and the like. When the terms “compound”, “agent”, “chemical agent” “pharmacologically active agent”, “medicament”, “active” and “drug” are used, then it is to be understood that this includes the active agent per se as well as pharmaceutically acceptable, pharmacologically active salts, esters, amides, prodrugs, metabolites, analogs, etc. The aforementioned compounds may specifically modulate expression of one or more differentially expressed genes (i.e. up-regulate or down-regulate expression as the case maybe) or they may modulate the activity of a gene product (i.e. increase or decrease the activity of a gene product) or they may replace an ineffective or low level of a gene product. Hence, the compounds contemplated herein may be useful in genetic therapy or in protein replacement or protein inhibitory therapy. Insofar as the compound is a genetic molecule, it may be DNA, RNA, an antisense molecule, a sense molecule, double stranded or single stranded RNA or DNA, short interfering RNA (siRNA), RNA interference (RNAi) or a complex of a nucleic acid and a ribonuclease.

Reference to a “compound”, “agent”, “chemical agent” “pharmacologically active agent”, “medicament”, “active” and “drug” includes combinations of two or more active agents. A “combination” also includes multi-part such as a two-part composition where the agents are provided separately and given or dispensed separately or admixed together prior to dispensation. For example, a multi-part pharmaceutical pack may have two or more agents separately maintained.

The terms “effective amount” and “therapeutically effective amount” of an agent as used herein mean a sufficient amount of the agent to provide the desired therapeutic or physiological effect or outcome. Such an effect or outcome includes modulating the expression or activity of a target gene or gene product or in the physiological outcome of intervention (such as amelioration of symptoms). Undesirable effects, e.g., side-effects, are sometimes manifested along with the desired therapeutic effect; hence, a practitioner balances the potential benefits against the potential risks in determining what is an appropriate “effective amount”. The exact amount required will vary from subject to subject, depending on the species, age and general condition of the subject, mode of administration and the like. Thus, it may not be possible to specify an exact “effective amount”. However, an appropriate “effective amount” in any individual case may be determined by one of ordinary skill in the art using only routine experimentation.

The “effective amount” also includes an amount to promote bone growth or overall health.

By “pharmaceutically acceptable” carrier, excipient or diluent is meant a pharmaceutical vehicle comprised of a material that is not biologically or otherwise undesirable, i.e. the material may be administered to a subject along with the selected active agent without causing any or a substantial adverse reaction. Carriers may include excipients and other additives such as diluents, detergents, coloring agents, wetting or emulsifying agents, pH buffering agents, preservatives and the like.

Similarly, a “pharmacologically acceptable” salt, ester, emide, prodrug or derivative of a compound as provided herein is a salt, ester, amide, prodrug or derivative that this not biologically or otherwise undesirable.

“Treating” a subject may involve prevention of a condition or other adverse physiological event in a susceptible individual as well as treatment of a clinically symptomatic individual by ameliorating the symptoms of the condition. In promoting overall bone health, or preventing bone damage, the term “prophylaxis” may also be used.

A “subject” as used herein refers to an animal, preferably a mammal and more preferably human who can benefit from the pharmaceutical formulations and methods of the present invention. There is no limitation on the type of animal that could benefit from the presently described pharmaceutical formulations and methods. A subject regardless of whether a human or non-human animal may be referred to as an individual, patient, animal, host or recipient. The compounds and methods of the present invention have applications in human medicine, veterinary medicine as well as in general, domestic or wild animal husbandry.

The terms “polypeptide”, “peptide” and “protein” are used interchangeably herein to refer to a polymer of amino acid residues. The terms apply to amino acid polymers in which one or more amino acid residue is an artificial chemical analog of a corresponding naturally occurring amino acid, naturally occurring amino acid polymers or recombinant polymers.

The term “target gene” or “target gene product” or “target protein” includes a gene or its expression product which is up-regulated or down-regulated in unfused versus fused sutures. A list of genes is provided in Tables 2, 3 and 4 which are encompassed by the term “target genes”. The expression products of these genes are examples of “target gene products”.

The term “antibody”, as used herein, includes various forms of modified or altered antibodies, such as an intact immunoglobulin, an Fv fragment containing only the light and heavy chain variable regions, an Fv fragment linked by a disulfide bond (Brinkmann et al, Proc. Natl, Acad. Sci. USA, 90:547-551, 1993), an Fab or (Fab)′2 fragment containing the variable regions and parts of the constant regions, a single-chain antibody and the like (Bird et al, Science 242:424-426, 1988; Huston et al, Proc. Nat. Acad. Sci. USA, 85:5879-5883, 1988). The antibody may be of animal (especially mouse, rat, sheep or goat) or human origin or may be chimeric (Morrison et al, Proc. Nat. Acad. Sci. USA, 81:6851-6855, 1984) or humanized (Jones et al, Nature 321:522-525, 1986).

The terms “nucleic acid” or “oligonucleotide” or grammatical equivalents herein refer to at least two nucleotides covalently linked together. A nucleic acid of the present invention is preferably single-stranded or double stranded and will generally contain phosphodiester bonds, although in some cases, as outlined below, nucleic acid analogs are included that may have alternate backbones, comprising, for example, phosphoramide (Beaucage et al, Tetrahedron 49(10):1925, 1993) and references therein; Letsinger, J. Org. Chem. 35:3800, 1970; Sprinzl et al, Eur. J. Biochem. 81:579, 1977; Letsinger et al, Nucl. Acids Res. 14:3487, 1986; Sawai et al, Chem. Lett. 805:1984; Letsinger et al, J. Am. Chem. Soc. 110:4470, 1988 and Pauwels et al, Chemica Scripta 26:1419, 1986), phosphorothioate (Mag et al, Nucleic Acids Res. 19:1437, 1991); U.S. Pat. No. 5,644,048 and phosphorodithioates (Briu et al, J. Am. Chem. Soc. 111:2321, 1989), O-methylphophoroamidite linkages (see Eckstein, Oligonucleotides and Analogues: A Practical Approach, Oxford University Press).

Other analog nucleic acids include those with positive backbones (Denpcy et al, Proc. Natl. Acad. Sci. USA, 92:6097, 1995); non-ionic backbones (U.S. Pat. Nos. 5,386,023; 5,637,684; 5,602,240; 5,216,141 and 4,469,863; Angew, Chem. Intl. Ed. English 30:423, 1991; Letsinger et al, 1988 supra; Letsinger et al, Nucleoside & Nucleotide 13:1597, 1994; Chapters 2 and 3, ASC Symposium Series 580, Carbohydrate Modifications in Antisense Research, Ed. Y S Sanghui and P Dan Cook; Mesmaeker et al, Bioorganic & Medicinal Chem. Lett. 4:395, 1994; Jeffs et al, J. Biomolecular NMR 34:17, 1994; Tetrahedron, Lett. 37:743, 1996) and non-ribose backbones, including those described in U.S. Pat. Nos. 5,235,033 and 5,034,506 and Chapters 6 and 7, ASC Symposium Series 580, Carbohydrate Modifications in Antisense Research, Ed. Y S Sanghui and P Dan Cook. Nucleic acids containing one or more carbocyclic sugars are also included within the definition of nucleic acids (Jenkins et al, Chem. Soc. Rev.:169-176, 1995). Several nucleic acid analogs are described in Rawls, C & E News:35, 1997. These modifications of the ribose-phosphate backbone may be done to facilitate the addition of additional moieties such as labels, or to increase the stability and half-life of such molecules in physiological environments.

The term “test agent” refers to an agent that is to be screened in one or more of the assays described herein. The agent can be virtually any chemical compound. It can exist as a single isolated compound or can be a member of a chemical (e.g. combinatorial) library. In a particularly preferred embodiment, the test agent will be a small organic molecule.

Again, the term “agent” may be replaced with “compound”, “molecule”, “medicament” and the like as listed above.

A “gene” includes a genomic gene or a cDNA molecule. The terms “gene”, “cDNA”, “nucleic acid molecule” and “nucleotide sequence” may be used interchangeably. A “nucleic acid molecule” may be RNA or DNA.

A “biomarker” may be the gene or gene product. A “gene product” may be a protein or RNA.

Hence, one aspect of the present invention contemplates a method for the treatment or prophylaxis of a bone pathology of reducing the risk of development of a bone pathology in a subject, said method comprising administering to said subject an effective amount of an agent which modulates expression of genetic material or the activity of encoded products of the genetic material wherein the genetic material is differentially expressed in unfused versus fused calvarial sutures.

The present invention further provides a method for promoting bone growth or health in a subject, said method comprising administering to said subject an effective amount of an agent which modulates expression of genetic material or the activity of encoded products of the genetic material wherein the genetic material is differentially expressed in unfused versus fused calvarial sutures.

Examples of a bone pathology are given above.

Accordingly, a particular embodiment of the present invention provides a method for the treatment or prophylaxis of a condition selected from one or more of bone cancer, a bone mineralization deficiency, fracture, a suture-based cranial abnormality, a cytoskeletal abnormality, osteoporosis or other biochemical or structural abnormality or condition, said method comprising administering to said subject an agent which modulates the level of expression of a gene or gene product which is up- or down-regulated in unfused sutures compared to fused sutures.

Examples of biomarkers for bone cancer include but are not limited to GPC3, RBP4, C1QTNF3, FMOD, WIF1, PRELP, PTN and CYFIP2 which have expression profiles shown in Table 5.

The present invention provides, therefore, a set of biomarkers comprising one or more genes or gene products differentially expressed in unfused sutures compared to fused sutures in a subject.

Examples of genes up-regulated in unfused sutures include but are not limited to MFAP4, RBP4, IL11RA, AMPH, INHBA, C1QTNF3, PRELP, FBLN1, ANGPTL2, AGC1, FMOD, OLFM1, C1orf24, AGC1, SSPN, PTN, MN1, TNN, EGFR, ADCY2, PDZRN3, SPON1, GPC3, HAPLN1, BCL11B, FLRT3, STXBP6, THBS2, KIAA0992, COL3A1, PAM, LSS, COL11A1, TOX, TRIM2, COL8A2, CRISPLD2, BHLHB3, EPHB2, DUSP10, COL11A1, OLFM1, TUBB2, SETBP1, ROR1, TGFB2, ISLR, PRSS11, COL16A1, S100A10, COL8A2, LOXL1, TRIM2, POSTN, LOXL2, CCND2, SSPN, ZCWCC2, ITGBL1, FLJ20701, PAM, ITGB5, MFAP2, CALD1, PTGER4, DIRAS3, THBS3, SIX2, COL6A1, ODZ3, AEBP1, EFEMP1, CAP2, DCAMKL1, ITGB5, CaMKIINalpha, JUN, SPON2, GAP43, EYA2, SNCAIP, EDG2, DNM1, PDZRN4, OGN, ASPN, MID1, ITGB5, EPHA4, RYR3, ATBF1, MEG3, HLF, OSBPL3, PDGFRL, DCHS1, GPC1, CDC42BPA, PTPRF, FGFR2, TLE2, COL6A3, FAT4, MMP14, MID1, LAMA2, LAMC1, EMX2, TMEFF1, PPP2R3A, ITM2A, MEG3, HS3ST3A1, RUNX1T1, PLAGL1, EPLIN, PLEKHA1, EGFL6, ARG2, MATN2, EDIL3, PCSK5, TGFB2, GULP1, MMP2, NELL2, PITX2, DACT1, DUSP10, NEDD4L, TMEM30B, TACC2, EWSR1, ITM2A, FGFR2, ANTXR1, PLCB1, MCC, HLF, TYRO3, FZD1, MT1X, NA, GULP1, OLFML1, ZFHX4, C3orf14, TAGLN, WASL, OSBPL3, SAMD4, CAPN6, FLJ12442, TGFB2, SEMA3 C, SPOCK, KCNK1, COL2A1, LAMA2, LMNA, GOLPH4, C14orf78, ARL7, ELOVL4, DPYSL3, TGFB3, COL10A1, PLOD3, GPNMB, COL14A1, TCF8, THY1, EHD2, PNMA2, MEIS2, DNCI1, FKBP14, RUNX1T1, COL6A2, RBM9, CCND1, NAP1L3, LRRN3, TIMP3, LOC133619, IGF1, GOLPH2, MAB21L2, PCLO, PRRX2, COL2A1, GDF10, PPP2R3A, PRSS23, SYNC1, IL6ST, LRRN3, PCSK5, NAV3, MAB21L2, GRP, FAP, GEM, EPHA3, MMP23B, TCEAL2, CREB5, KAL1, HSPA1B, FLJ10970, TPSAB1, SCRG1, TBC1D19, TPSB2, HCFC1R1, TPSAB1, SC65, ATF3, CART1, WIF1 and HLA-DRB1.

Examples of genes up-regulated in fused sutures include but are not limited to CYFIP2, ABCG1, ANAXA3, FABP4, DPYD, RNASE6, CHD7, HIST1H1E, CASP1, FLI1, ATF7IP2, ST6GAL1, MMD, LOC54103, PTPRE, PTPN22, TNFSF10, RAB11FIP1, MYCBP, RASSF2, SCAP2, HHEX, CD163, C18orf1, RAB27A, LOC54103, IL7R, GPR126, P2RY14, ARHGAP15, FCER1G, RGS2, HLA-DMB, PLSCR1, TRIM22, FAM60A, CD300A, BLM, PARP8, LAPTM5, CG018, LIG4, RAC2, RAB20, LOC93349, GENX-3414, E2 F5, DKFZP586A0522, ACSL1, OAS2, IQGAP2, CLEC2D, HLA-DMA, ZNF588, CD53, SLC4A4, TACSTD1, CXCR4, MFAP3L, TLR2, LCP2, LRMP, IL8RB, ARHGAP19, CXCR4, C1orf38, FLJ11127, LY75, HLA-DRA, MAPK14, PTPRC, SLA, ENPP4, PLCG2, PLEKHF2, STX3A, CENTD1, RIF1, PTPRC, VWF, TTF2, CAPN3, TARP, PRKAR2B, EVI2B, GPLD1, HLA-DQB1, OLR1, NCB5OR, NPL, SLC15A2, TPD52, LCP1, HEM1, PSMB8, RHOH, FCGR3B, KIAA0125, SOCS2, CDC7, QRSL1, SCAP2, BIN2, GMFG, WBSCR5, PYGL, RRAGD, CXCR4, BCL2A1, GZMB, PSMB9, SMC2L1, LY64, ME2, MCM10, AMPD3, IL18RAP, P2RY13, IGHG1, HMOX1, TKT, SLCO4C1, IGLC2, PSCDBP, PRKCB1, LRMP, GAS7, ORM1, CCR2, CRISP2, HERC5, OIP5, SCAP2, GNG4, POLQ, HLA-DPA1, MS4A4A, DC12, FLJ22662, ITK, GIT2, TPD52, SYK, GCH1, ORM1, IMP-3, CD69, MME, ZNFN1A1, MCTP2, MS4A1, RAC2, CEACAM1, ATP8B4, ISG20, TAL1, CD38, RAG2, CUGBP2, BLNK, IMPA2, CRHBP, PLK4, ME2, ARHGDIB, TNFRSF17, BRRN1, CD74, AIF1, MONDOA, CCNA2, CLC, S100A12, CEACAM8, PLAC8, ANXA3, SORL1, LTF, POU2AF1, LCN2, OLFM4, CRISP3, MPO, TCL1A, MPO, DNTT, PRTN3, S100A9, MS4A3, RNASE3, MMP8, MNDA, SELL, ALOX5, HP, SNCA, CAMP, FCN1, ARG1, CEACAM6, GCA, MYB, RNASE2, IGHM, IRF4, RAG1, FCGR3B, TCN1, TCL1A, CORO1A, SPTA1, CEACAM6, PADI4, CSTA, PF4, GYPA, CD37, S100P, NCF2, PRG1, ALAS2, HLA-DQB1, CYP4F3, ALOX5AP, MGAM, IGLL1, IGHM, C13orf18, VPREB1, HBG2 and CHI3L1.

Particularly useful biomarkers include but are not limited to COL8A2, PRELP, RBP4, C1QTNF3, GPC3, CYFIP2, MFAP4, AMPH, IL11RA, INHBA, WIF1, ANXA3, CASP1, SHOX2 and PTN.

Even more particularly useful genes include but are not limited to GPC3, RBP4, C1QTNF3, ANAXA3, WIF1 and CASP1. GPC3, RBP4 and C1QTNF3 are highly expressed in unfused sutures whereas WIF1 and CASP1 are highly expressed in fused and fusing sutures.

Reference to the above genes include polymorphic variants mutants and derivatives thereof as well as homologs thereof.

Accordingly, another aspect of the present invention contemplates a method of treating or reducing the risk of development of a bone pathology in a subject, said method comprising administering to said subject an agent which down-regulates a gene or gene product selected from the list comprising MFAP4, RBP4, IL11RA, AMPH, INHBA, C1QTNF3, PRELP, FBLN1, ANGPTL2, AGC1, FMOD, OLFM1, C1orf24, AGC1, SSPN, PTN, MN1, TNN, EGFR, ADCY2, PDZRN3, SPON1, GPC3, HAPLN1, BCL11B, FLRT3, STXBP6, THBS2, KIAA0992, COL3A1, PAM, LSS, COL11A1, TOX, TRIM2, COL8A2, CRISPLD2, BHLHB3, EPHB2, DUSP10, COL11A1, OLFM1, TUBB2, SETBP1, ROR1, TGFB2, ISLR, PRSS11, COL16A1, S100A10, COL8A2, LOXL1, TRIM2, POSTN, LOXL2, CCND2, SSPN, ZCWCC2, ITGBL1, FLJ20701, PAM, ITGB5, MFAP2, CALD1, PTGER4, DIRAS3, THBS3, SIX2, COL6A1, ODZ3, AEBP1, EFEMP1, CAP2, DCAMKL1, ITGB5, CaMKIINalpha, JUN, SPON2, GAP43, EYA2, SNCAIP, EDG2, DNM1, PDZRN4, OGN, ASPN, MID1, ITGB5, EPHA4, RYR3, ATBF1, MEG3, HLF, OSBPL3, PDGFRL, DCHS1, GPC1, CDC42BPA, PTPRF, FGFR2, TLE2, COL6A3, FAT4, MMP14, MID1, LAMA2, LAMC1, EMX2, TMEFF1, PPP2R3A, ITM2A, MEG3, HS3ST3A1, RUNX1T1, PLAGL1, EPLIN, PLEKHA1, EGFL6, ARG2, MATN2, EDIL3, PCSK5, TGFB2, GULP1, MMP2, NELL2, PITX2, DACT1, DUSP10, NEDD4L, TMEM30B, TACC2, EWSR1, ITM2A, FGFR2, ANTXR1, PLCB1, MCC, HLF, TYRO3, FZD1, MT1X, NA, GULP1, OLFML1, ZFHX4, C3orf14, TAGLN, WASL, OSBPL3, SAMD4, CAPN6, FLJ12442, TGFB2, SEMA3C, SPOCK, KCNK1, COL2A1, LAMA2, LMNA, GOLPH4, C14orf78, ARL7, ELOVL4, DPYSL3, TGFB3, COL10A1, PLOD3, GPNMB, COL14A1, TCF8, THY1, EHD2, PNMA2, MEIS2, DNCI1, FKBP14, RUNX1T1, COL6A2, RBM9, CCND1, NAP1L3, LRRN3, TIMP3, LOC133619, IGF1, GOLPH2, MAB21L2, PCLO, PRRX2, COL2A1, GDF10, PPP2R3A, PRSS23, SYNC1, IL6ST, LRRN3, PCSK5, NAV3, MAB21L2, GRP, FAP, GEM, EPHA3, MMP23B, TCEAL2, CREB5, KAL1, HSPA1B, FLJ10970, TPSAB1, SCRG1, TBC1D19, TPSB2, HCFC1R1, TPSAB1, SC65, ATF3, CART1, WIF1 and HLA-DRB1 or which up-regulates expression of a gene or gene product selected from the list comprising CYFIP2, ABCG1, FABP4, ANAXA3, DPYD, RNASE6, CHD7, HIST1H1E, CASP1, FLI1, ATF7IP2, ST6GAL1, MMD, LOC54103, PTPRE, PTPN22, TNFSF10, RAB11FIP1, MYCBP, RASSF2, SCAP2, HHEX, CD163, C18orf1, RAB27A, LOC54103, IL7R, GPR126, P2RY14, ARHGAP15, CASP1, FCER1G, RGS2, HLA-DMB, PLSCR1, TRIM22, FAM60A, CD300A, BLM, PARP8, LAPTM5, CG018, LIG4, RAC2, RAB20, LOC93349, GENX-3414, E2F5, DKFZP586A0522, ACSL1, OAS2, IQGAP2, CLEC2D, HLA-DMA, ZNF588, CD53, SLC4A4, TACSTD1, CXCR4, MFAP3L, TLR2, LCP2, LRMP, IL8RB, ARHGAP19, CXCR4, C1orf38, FLJ11127, LY75, HLA-DRA, MAPK14, PTPRC, SLA, ENPP4, PLCG2, PLEKHF2, STX3A, CENTD1, RIF1, PTPRC, VWF, TTF2, CAPN3, TARP, PRKAR2B, EVI2B, GPLD1, HLA-DQB1, OLR1, NCB5OR, NPL, SLC15A2, TPD52, LCP1, HEM1, PSMB8, RHOH, FCGR3B, KIAA0125, SOCS2, CDC7, QRSL1, SCAP2, BIN2, GMFG, WBSCR5, PYGL, RRAGD, CXCR4, BCL2A1, GZMB, PSMB9, SMC2L1, LY64, ME2, MCM10, AMPD3, IL18RAP, P2RY13, IGHG1, HMOX1, TKT, SLCO4C1, IGLC2, PSCDBP, PRKCB1, LRMP, GAS7, ORM1, CCR2, CRISP2, HERC5, OIP5, SCAP2, GNG4, POLQ, HLA-DPA1, MS4A4A, DC12, FLJ22662, ITK, GIT2, TPD52, SYK, GCH1, ORM1, IMP-3, CD69, MME, ZNFN1A1, MCTP2, MS4A1, RAC2, CEACAM1, ATP8B4, ISG20, TAL1, CD38, RAG2, CUGBP2, BLNK, IMPA2, CRHBP, PLK4, ME2, ARHGDIB, TNFRSF17, BRRN1, CD74, AIF1, MONDOA, CCNA2, CLC, S100A12, CEACAM8, PLACE, ANXA3, SORL1, LTF, POU2AF1, LCN2, OLFM4, CRISP3, MPO, TCL1A, MPO, DNTT, PRTN3, S100A9, MS4A3, RNASE3, MMP8, MNDA, SELL, ALOX5, HP, SNCA, CAMP, FCN1, ARG1, CEACAM6, GCA, MYB, RNASE2, IGHM, IRF4, RAG1, FCGR3B, TCN1, TCL1A, CORO1A, SPTA1, CEACAM6, PADI4, CSTA, PF4, GYPA, CD37, S1OOP, NCF2, PRG1, ALAS2, HLA-DQB1, CYP4F3, ALOX5AP, MGAM, IGLL1, IGHM, C13orf18, VPREB1, HBG2 and CHI3L1.

The up-regulation of the above genes is at least 2-fold higher than controls and at least up to 100-fold such as 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100-fold up-regulation.

A value of from at least about 2-fold to at least about 40-fold up-regulation is preferred and a value of from at least about 10-fold to about 40-fold is particularly preferred.

The present invention also provides a genetic construct comprising an encoding nucleic acid molecule or an antisense version thereof or which otherwise targets a nucleic acid molecule selected from the list comprising a nucleic acid molecule whose expression is up-regulated or down-regulated in unfused versus fused sutures such as a gene selected from MFAP4, RBP4, IL11RA, AMPH, INHBA, C1QTNF3, PRELP, FBLN1, ANGPTL2, AGC1, FMOD, OLFM1, C1orf24, AGC1, SSPN, PTN, MN1, TNN, EGFR, ADCY2, PDZRN3, SPON1, GPC3, HAPLN1, BCL11B, FLRT3, STXBP6, THBS2, KIAA0992, COL3A1, PAM, LSS, COL11A1, TOX, TRIM2, COL8A2, CRISPLD2, BHLHB3, EPHB2, DUSP10, COL11A1, OLFM1, TUBB2, SETBP1, ROR1, TGFB2, ISLR, PRSS11, COL16A1, S100A10, COL8A2, LOXL1, TRIM2, POSTN, LOXL2, CCND2, SSPN, ZCWCC2, ITGBL1, FLJ20701, PAM, ITGB5, MFAP2, CALD1, PTGER4, DIRAS3, THBS3, SIX2, COL6A1, ODZ3, AEBP1, EFEMP1, CAP2, DCAMKL1, ITGB5, CaMKIINalpha, JUN, SPON2, GAP43, EYA2, SNCAIP, EDG2, DNM1, PDZRN4, OGN, ASPN, MID1, ITGB5, EPHA4, RYR3, ATBF1, MEG3, HLF, OSBPL3, PDGFRL, DCHS1, GPC1, CDC42BPA, PTPRF, FGFR2, TLE2, COL6A3, FAT4, MMP14, MID1, LAMA2, LAMC1, EMX2, TMEFF1, PPP2R3A, ITM2A, MEG3, HS3ST3A1, RUNX1T1, PLAGL1, EPLIN, PLEKHA1, EGFL6, ARG2, MATN2, EDIL3, PCSK5, TGFB2, GULP1, MMP2, NELL2, PITX2, DACT1, DUSP10, NEDD4L, TMEM30B, TACC2, EWSR1, ITM2A, FGFR2, ANTXR1, PLCB1, MCC, HLF, TYRO3, FZD1, MT1X, NA, GULP1, OLFML1, ZFHX4, C3orf14, TAGLN, WASL, OSBPL3, SAMD4, CAPN6, FLJ12442, TGFB2, SEMA3C, SPOCK, KCNK1, COL2A1, LAMA2, LMNA, GOLPH4, C14orf78, ARL7, ELOVL4, DPYSL3, TGFB3, COL10A1, PLOD3, GPNMB, COL14A1, TCF8, THY1, EHD2, PNMA2, MEIS2, DNCI1, FKBP14, RUNX1T1, COL6A2, RBM9, CCND1, NAP1L3, LRRN3, TIMP3, LOC133619, IGF1, GOLPH2, MAB21L2, PCLO, PRRX2, COL2A1, GDF10, PPP2R3A, PRSS23, SYNC1, IL6ST, LRRN3, PCSK5, NAV3, MAB21L2, GRP, FAP, GEM, EPHA3, MMP23B, TCEAL2, CREB5, KAL1, HSPA1B, FLJ10970, TPSAB1, SCRG1, TBC1D19, TPSB2, HCFC1R1, TPSAB1, SC65, ATF3, CART1, WIF1, HLA-DRB1, CYFIP2, ABCG1, FABP4, DPYD, ANAXA3, RNASE6, CHD7, HIST1H1E, CASP1, FLI1, ATF7IP2, ST6GAL1, MMD, LOC54103, PTPRE, PTPN22, TNFSF10, RAB11FIP1, MYCBP, RASSF2, SCAP2, HHEX, CD163, C18orf1, RAB27A, LOC54103, IL7R, GPR126, P2RY14, ARHGAP15, CASP1, FCER1G, RGS2, HLA-DMB, PLSCR1, TRIM22, FAM60A, CD300A, BLM, PARP8, LAPTM5, CG018, LIG4, RAC2, RAB20, LOC93349, GENX-3414, E2F5, DKFZP586A0522, ACSL1, OAS2, IQGAP2, CLEC2D, HLA-DMA, ZNF588, CD53, SLC4A4, TACSTD1, CXCR4, MFAP3L, TLR2, LCP2, LRMP, IL8RB, ARHGAP19, CXCR4, C1orf38, FLJ11127, LY75, HLA-DRA, MAPK14, PTPRC, SLA, ENPP4, PLCG2, PLEKHF2, STX3A, CENTD1, RIF1, PTPRC, VWF, TTF2, CAPN3, TARP, PRKAR2B, EVI2B, GPLD1, HLA-DQB1, OLR1, NCB5OR, NPL, SLC15A2, TPD52, LCP1, HEM1, PSMB8, RHOH, FCGR3B, KIAA0125, SOCS2, CDC7, QRSL1, SCAP2, BIN2, GMFG, WBSCR5, PYGL, RRAGD, CXCR4, BCL2A1, GZMB, PSMB9, SMC2L1, LY64, ME2, MCM10, AMPD3, IL18RAP, P2RY13, IGHG1, HMOX1, TKT, SLCO4C1, IGLC2, PSCDBP, PRKCB1, LRMP, GAS7, ORM1, CCR2, CRISP2, HERC5, OIP5, SCAP2, GNG4, POLQ, HLA-DPA1, MS4A4A, DC12, FLJ22662, ITK, GIT2, TPD52, SYK, GCH1, ORM1, IMP-3, CD69, MME, ZNFN1A1, MCTP2, MS4A1, RAC2, CEACAM1, ATP8B4, ISG20, TAL1, CD38, RAG2, CUGBP2, BLNK, IMPA2, CRHBP, PLK4, ME2, ARHGDIB, TNFRSF17, BRRN1, CD74, AIF1, MONDOA, CCNA2, CLC, S100A12, CEACAM8, PLACE, ANXA3, SORL1, LTF, POU2AF1, LCN2, OLFM4, CRISP3, MPO, TCL1A, MPO, DNTT, PRTN3, S100A9, MS4A3, RNASE3, MMP8, MNDA, SELL, ALOX5, HP, SNCA, CAMP, FCN1, ARG1, CEACAM6, GCA, MYB, RNASE2, IGHM, IRF4, RAG1, FCGR3B, TCN1, TCL1A, CORO1A, SPTA1, CEACAM6, PADI4, CSTA, PF4, GYPA, CD37, S1OOP, NCF2, PRG1, ALAS2, HLA-DQB1, CYP4F3, ALOX5AP, MGAM, IGLL1, IGHM, C13orf18, VPREB1, HBG2 and CHI3L1.

Particularly useful target genes are COL8A2, PRELP, RBP4, C1QTNF3, CYFIP2, MFAP4, AMPH, IL11RA, INHBA, WIF1, ANXA3, CASP1, SHOX2 and PTN.

Even more particularly useful target genes are GPC3, RBP4, C1QTNF3, ANAXA3, WIF1 and CASP1.

The identification of differentially expressed genes associated with the bone morphologies enable therapeutic and diagnostic protocols to be developed. Therapeutic protocols encompass manipulation of gene expression, gene replacement and modulation of protein activity or protein replacement therapy. Diagnostic protocols include genetic and protein based assays aimed at determining the level of gene expression or gene products and/or the presence of any mutations in the genes or gene products.

The present invention further provides, therefore, the use of one or more genes or gene products differentially expressed in unfused sutures compared to fused sutures in a subject in the manufacture of a medicament for the treatment of a bone pathology.

The differential expression is conveniently determined in patients with craniosynostosis.

Hence, the present invention contemplates targeting genes whose aberrant expression leads to premature fusion of sutures but which may also be associated with other bone pathologies as listed above.

Hence, in still another embodiment the present invention provides a method of screening for an agent which modulates the level of activity of a target gene or target gene product associated with a bone pathology. The method includes contacting a test cell containing a target gene with a test agent and detecting a change in the expression level of the target gene or the activity of target gene product in the test cell as compared to the expression of the target gene or the activity of the target gene product in a control cell where a difference in expression level of the target gene or the activity of the target gene product in the test cell and the control cell indicates that said agent may modulate the symptoms of a bone pathology. In certain embodiments, the control is a negative control cell contacted with the test agent at a lower concentration than the test cell. In various embodiments, the expression level of the target gene is detected by measuring the level of the target gene mRNA in said cell and/or the level of target gene product is detected by determining the level of protein in the biological cell.

The present invention provides, therefore, therapeutic agents which interact with a target gene, target gene transcript or other gene product (such as a protein). There are several steps commonly taken in the design of such therapeutic agents. First, the particular parts of the target critical for expression or activity are determined. In the case of a protein, for example, this can be done by systematically varying the amino acid residues in the peptide, e.g. by substituting each residue in turn. Alanine scans of proteins, for example, are commonly used to define such protein motifs. These parts or residues constituting the active region of the compound are known as its “pharmacophore”. As indicated above, the terms “peptide”, “polypeptide” or “protein” may be used interchangeably.

Once the pharmacophore has been found, its structure is modeled according to its physical properties, e.g. stereochemistry, bonding, size and/or charge, using data from a range of sources, e.g. spectroscopic techniques, x-ray diffraction data and NMR. Computational analysis, similarity mapping (which models the charge and/or volume of a pharmacophore, rather than the bonding between atoms) and other techniques can be used in this modeling process.

In a variant of this approach, the three-dimensional structure of a target is modeled. Modeling can be used to generate agents which interact with the linear sequence or a three-dimensional configuration.

A template molecule is then selected onto which chemical groups which mimic the pharmacophore can be grafted. The template molecule and the chemical groups grafted onto it can conveniently be selected so that the therapeutic agent is easy to synthesize, is likely to be pharmacologically acceptable, and does not degrade in vivo, while retaining the biological activity of the lead compound. Alternatively, where the agent is peptide-based, further stability can be achieved by cyclizing the peptide, increasing its rigidity. The agents found by this approach can then be screened to see whether they have the target property, or to what extent they can modulate the activity of a target protein or modulation expression of a target gene. Further optimization or modification can then be carried out to arrive at one or more final agents for in vivo or clinical testing.

The goal of rational drug design is to produce structural analogs or antagonists of biologically active polypeptides of interest or of small molecules with which they interact (e.g. agonists, antagonists, inhibitors or enhancers) in order to fashion drugs which are, for example, more active or stable forms of the polypeptide, or which, for example, enhance or interfere with the function of a polypeptide in vivo (see, e.g. Hodgson, BioTechnology 9:19-21, 1991).

Agents are also contemplated by the present invention which regulate expression of target genes. This could involve, inter alia, providing gene function to a cell such as in gene therapy, or, it could involve inhibiting gene function using gene silencing constructs including antisense oligonucleotides or expression constructs.

A target nucleic acid sequence or a part of a nucleic acid sequence, such as a nucleic acid sequence capable of regulating nucleic acid expression may be introduced into a cell in a vector such that the nucleic acid sequence remains extrachromosomal. In such a situation, the nucleic acid sequence will be expressed by the cell from the extrachromosomal location. Vectors for introduction of nucleic acid sequence both for recombination and for extrachromosomal maintenance are known in the art and any suitable vector may be used. Methods for introducing nucleic acids into cells such as electroporation, calcium phosphate co-precipitation and viral transduction are known in the art.

In particular, a number of viruses have been used as nucleic acid transfer vectors or as the basis for preparing nucleic acid transfer vectors, including papovaviruses (e.g. SV40, Madzak et al, J Gen Virol 73:1533-1536, 1992), adenovirus (Berkner, Curr Top Microbiol Immunol 158:39-66, 1992; Berkner et al, BioTechniques 6:616-629, 1988; Gorziglia and Kapikian, J Virol 66:4407-4412, 1992; Quantin et al, Proc Natl Acad Sci USA 89:2581-2584, 1992; Rosenfeld et al, Cell 68:143-155, 1992; Wilkinson et al, Nucleic Acids Res 20:233-2239, 1992; Stratford-Perricaudet et al, Hum Gene Ther 1:241-256, 1990; Schneider et al, Nat Genetics 18:180-183, 1998), vaccinia virus (Moss, Curr Top Microbiol Immunol 158: 5-38, 1992; Moss, Proc Natl Acad Sci USA 93:11341-11348, 1996), adeno-associated virus (Muzyczka, Curr Top Microbiol Immunol 158:97-129, 1992; Ohi et al, Gene 89:279-282, 1990; Russell and Hirata, Nat Genetics 18:323-328, 1998), herpesviruses including HSV and EBV (Margolskee, Curr Top Microbiol Immunol 158:67-95, 1992; Johnson et al, J Virol 66:2952-2965, 1992; Fink et al, Hum Gene Ther 3:1-19, 1992; Breakefield and Geller, Mol Neurobiol 1:339-371, 1987; Freese et al, Biochem Pharmaco. 40:2189-2199, 1990; Fink et al, Ann Rev Neurosci 19:265-287, 1996), lentiviruses (Naldini et al, Science 272:263-267, 1996), Sindbis and Semliki Forest virus (Berglund et al, Biotechnology 11:916-920, 1993) and retroviruses of avian (Bandyopadhyay and Temin, Mol Cell Biol 4:749-754, 1984; Petropoulos et al, J Virol 66:3391-3397, 1992), murine (Miller, Curr Top Microbiol Immunol 158:1-24, 1992; Miller et al, Mol Cell Biol 5:431-437, 1985; Sorge et al, Mol Cell Biol 4:1730-1737, 1984; Mann and Baltimore, J Virol 54:401-407, 1985; Miller et al, J Virol 62:4337-4345, 1988) and human (Shimada et al, J Clin Invest 88:1043-1047, 1991; Helseth et al, J Virol 64:2416-2420, 1990; Page et al, J Virol 64:5270-5276, 1990; Buchschacher and Panganiban, J Virol 66:2731-2739, 1982) origin.

Non-viral nucleic acid transfer methods are known in the art such as chemical techniques including calcium phosphate co-precipitation, mechanical techniques, for example, microinjection, membrane fusion-mediated transfer via liposomes and direct DNA uptake and receptor-mediated DNA transfer. Viral-mediated nucleic acid transfer can be combined with direct in vivo nucleic acid transfer using liposome delivery, allowing one to direct the viral vectors to particular cells. Alternatively, the retroviral vector producer cell line can be injected into particular tissue. Injection of producer cells would then provide a continuous source of vector particles.

The present invention further contemplates the introduction of antisense and sense molecules such as polynucleotide sequences, which are useful in silencing transcripts of target genes. Ribozymes, micro RNAs, synthetic RNAi, DNA-derived RNAi as well as double stranded RNAs may also be introduced. Both pre-transcriptional and post-transcriptional gene silencing is contemplated including antisense silencing. Furthermore, polynucleotide vectors containing all or a portion of a gene locus encoding the expression product of a target gene may be placed under the control of a promoter in an antisense or sense orientation and introduced into a cell. Expression of such an antisense or sense construct within a cell interferes with target transcription and/or translation.

In one embodiment, the engineered genetic molecules encode oligonucleotides and similar species for use in modulating the expression of target genes, i.e. the oligonucleotides induce pre-transcriptional or post-transcriptional gene silencing. This is accomplished by providing oligonucleotides which specifically hybridize to or otherwise target one or more target nucleic acid molecules encoding the target gene product. Hence, the constructs may encode inter alia micro RNA, dsRNA, hairpin RNAs, RNAi, siRNA or DNA. As used herein, the term “target gene” is used for convenience to encompass DNA encoding the target gene product, RNA (including pre-mRNA and mRNA or portions thereof) transcribed from such DNA, and also cDNA derived from such RNA.

In another embodiment, therefore, the present invention provides a method for treatment or prophylaxis of diseases or conditions characterized by being or causing a bone pathology comprising administering to a subject an agent capable of regulating expression of a gene which is differentially expressed in un-fused sutures versus fused sutures. This method includes promoting bone growth or overall health. As indicated above a non-genetic therapeutic agent may be administered. The agents of the present invention can be combined with one or more pharmaceutically acceptable carriers and/or diluents to form a pharmacological composition. Pharmaceutically acceptable carriers can contain a physiologically acceptable compound that acts to, e.g., stabilize, or increase or decrease the absorption or clearance rates of the pharmaceutical compositions of the invention. Physiologically acceptable compounds can include, e.g., carbohydrates, such as glucose, sucrose, or dextrans, antioxidants, such as ascorbic acid or glutathione, chelating agents, low molecular weight proteins, compositions that reduce the clearance or hydrolysis of the peptides or polypeptides, or excipients or other stabilizers and/or buffers. Detergents can also be used to stabilize or to increase or decrease the absorption of the pharmaceutical composition, including liposomal carriers. Pharmaceutically acceptable carriers and formulations for peptides and polypeptide are known to the skilled artisan and are described in detail in the scientific and patent literature, see e.g., Remington's Pharmaceutical Sciences, 18^{th} Edition, Mack Publishing Company, Easton, Pa., 1990 (“Remington's”).

Other physiologically acceptable compounds include wetting agents, emulsifying agents, dispersing agents or preservatives which are particularly useful for preventing the growth or action of microorganisms. Various preservatives are well known and include, e.g., phenol and ascorbic acid. One skilled in the art would appreciate that the choice of a pharmaceutically acceptable carrier including a physiologically acceptable compound depends, for example, on the route of administration of the modulatory agent of the invention and on its particular physio-chemical characteristics.

Administration of the agent, in the form of a pharmaceutical composition, may be performed by any convenient means known to one skilled in the art. Routes of administration include, but are not limited to, respiratorally, intratracheally, nasopharyngeally, intravenously, intraperitoneally, subcutaneously, intracranially, intradermally, intramuscularly, intraoccularly, intrathecally, intracereberally, intranasally, orally, rectally, patch and implant.

For oral administration, the compounds can be formulated into solid or liquid preparations such as capsules, pills, tablets, lozenges, powders, suspensions or emulsions. In preparing the compositions in oral dosage form, any of the usual pharmaceutical media may be employed, such as, for example, water, glycols, oils, alcohols, flavoring agents, preservatives, coloring agents, suspending agents, and the like in the case of oral liquid preparations (such as, for example, suspensions, elixirs and solutions); or carriers such as starches, sugars, diluents, granulating agents, lubricants, binders, disintegrating agents and the like in the case of oral solid preparations (such as, for example, powders, capsules and tablets). Due to their ease in administration, tablets and capsules represent the most advantageous oral dosage unit form, in which case solid pharmaceutical carriers are obviously employed. If desired, tablets may be sugar-coated or enteric-coated by standard techniques. The active agent can be encapsulated to make it stable to passage through the gastrointestinal tract while at the same time allowing for passage across the blood brain barrier, see, e.g, International Patent Publication Number WO 96/11698.

Agents of the present invention, when administered orally, may be protected from digestion. This can be accomplished either by complexing the agent with a composition to render it resistant to acidic and enzymatic hydrolysis or by packaging the agent in an appropriately resistant carrier such as a liposome. Means of protecting compounds from digestion are well known in the art, see, e.g. Fix, Pharm Res 13:1760-1764, 1996; Samanen et al, J Pharm Pharmacol 48:119-135, 1996; U.S. Pat. No. 5,391,377, describing lipid compositions for oral delivery of therapeutic agents.

The pharmaceutical forms suitable for injectable use include sterile aqueous solutions (where water-soluble) or dispersions and sterile powders for the preparation of sterile injectable solutions or dispersion or may be in the form of a cream or other form suitable for topical application. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of superfactants. The prevention of the action of microorganisms can be brought about by various antibacterial and antifungal agents, for example, parabens, chlorobutanol, phenol, sorbic acid, thimerosal and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the agents in the required amount in the appropriate solvent with various of the other ingredients enumerated above, as required, followed by filtered sterilization. Generally, dispersions are prepared by incorporating the various sterilised active ingredient into a sterile vehicle which contains the basic dispersion medium and the required other ingredients from those enumerated above. In the case of sterile powders for the preparation of sterile injectable solutions, the preferred methods of preparation are vacuum drying and the freeze-drying technique which yield a powder of the active ingredient plus any additional desired ingredient from previously sterile-filtered solution thereof.

For parenteral administration, the agent may be dissolved in a pharmaceutical carrier and administered as either a solution or a suspension. Illustrative of suitable carriers are water, saline, dextrose solutions, fructose solutions, ethanol, or oils of animal, vegetative or synthetic origin. The carrier may also contain other ingredients, for example, preservatives, suspending agents, solubilizing agents, buffers and the like. When the agents are being administered intrathecally, they may also be dissolved in cerebrospinal fluid.

For transmucosal or transdermal administration, penetrants appropriate to the barrier to be permeated can be used for delivering the agent. Such penetrants are generally known in the art e.g. for transmucosal administration, bile salts and fusidic acid derivatives. In addition, detergents can be used to facilitate permeation. Transmucosal administration can be through nasal sprays or using suppositories e.g. Sayani and Chien, Crit Rev Ther Drug Carrier Syst 13:85-184, 1996. For topical, transdermal administration, the agents are formulated into ointments, creams, salves, powders and gels. Transdermal delivery systems can also include patches.

For inhalation, the agents of the invention can be delivered using any system known in the art, including dry powder aerosols, liquids delivery systems, air jet nebulizers, propellant systems, and the like, see, e.g., Patton, Nat Biotech 16:141-143, 1998; product and inhalation delivery systems for polypeptide macromolecules by, e.g., Dura Pharmaceuticals (San Diego, Calif.), Aradigm (Hayward, Calif.), Aerogen (Santa Clara, Calif.), Inhale Therapeutic Systems (San Carlos, Calif.), and the like. For example, the pharmaceutical formulation can be administered in the form of an aerosol or mist. For aerosol administration, the formulation can be supplied in finely divided form along with a surfactant and propellant. In another aspect, the device for delivering the formulation to respiratory tissue is an inhaler in which the formulation vaporizes. Other liquid delivery systems include, for example, air jet nebulizers.

The agents of the subject invention can also be administered in sustained delivery or sustained release mechanisms, which can deliver the formulation internally. For example, biodegradable microspheres or capsules or other biodegradable polymer configurations capable of sustained delivery of an agent can be included in the formulations of the instant invention (e.g. Putney and Burke, Nat Biotech 16:153-157, 1998).

In preparing pharmaceuticals of the present invention, a variety of formulation modifications can be used and manipulated to alter pharmacokinetics and biodistribution. A number of methods for altering pharmacokinetics and biodistribution are known to one of ordinary skill in the art. Examples of such methods include protection of the compositions of the invention in vesicles composed of substances such as proteins, lipids (for example, liposomes), carbohydrates, or synthetic polymers. For a general discussion of pharmacokinetics, see, e.g., Remington's.

In one aspect, the pharmaceutical formulations comprising agents of the present invention are incorporated in lipid monolayers or bilayers such as liposomes, see, e.g., U.S. Pat. Nos. 6,110,490; 6,096,716; 5,283,185 and 5,279,833. The invention also provides formulations in which water-soluble modulatory agents of the invention have been attached to the surface of the monolayer or bilayer. For example, peptides can be attached to hydrazide-PEG-(distearoylphosphatidyl)ethanolamine-containing liposomes (e.g. Zalipsky et al, Bioconjug Chem 6:705-708, 1995). Liposomes or any form of lipid membrane, such as planar lipid membranes or the cell membrane of an intact cell e.g. a red blood cell, can be used. Liposomal formulations can be by any means, including administration intravenously, transdermally (Vutla et al, J Pharm Sci 85:5-8, 1996), transmucosally, or orally. The invention also provides pharmaceutical preparations in which the agents of the invention are incorporated within micelles and/or liposomes (Suntres and Shek, J Pharm Pharmacol 46:23-28, 1994; Woodle et al, Pharm Res 9:260-265, 1992). Liposomes and liposomal formulations can be prepared according to standard methods and are also well known in the art see, e.g., Remington's; Akimaru et al, Cytokines Mol Ther 1:197-210, 1995; Alving et al, Immunol Rev 145:5-31, 1995; Szoka and Papahadjopoulos, Ann Rev Biophys Bioeng 9:467-508, 1980, U.S. Pat. Nos. 4,235,871, 4,501,728 and 4,837,028.

The pharmaceutical compositions of the invention can be administered in a variety of unit dosage forms depending upon the method of administration. Dosages for typical pharmaceutical compositions are well known to those of skill in the art. Such dosages are typically advisorial in nature and are adjusted depending on the particular therapeutic context, patient tolerance, etc. The amount of agent adequate to accomplish this is defined as the “effective amount”. The dosage schedule and effective amounts for this use, i.e. the “dosing regimen” will depend upon a variety of factors, including the stage of the disease or condition, the severity of the disease or condition, the general state of the patient's health, the patient's physical status, age, pharmaceutical formulation and concentration of active agent, and the like. In calculating the dosage regimen for a patient, the mode of administration also is taken into consideration. The dosage regimen must also take into consideration the pharmacokinetics, i.e. the pharmaceutical composition's rate of absorption, bioavailability, metabolism, clearance, and the like. See, e.g., Remington's; Egleton and Davis, Peptides 18:1431-1439, 1997; Langer, Science 249:1527-1533, 1990.

In accordance with these methods, the agents and/or pharmaceutical compositions defined in accordance with the present invention may be co-administered with one or more other agents. Reference herein to “co-administered” means simultaneous administration in the same formulation or in two different formulations via the same or different routes or sequential administration by the same or different routes. Reference herein to “sequential” administration is meant a time difference of from seconds, minutes, hours or days between the administration of the two types of agents and/or pharmaceutical compositions. Co-administration of the agents and/or pharmaceutical compositions may occur in any order.

The present invention also facilitates the development of diagnostic and/or prognostic assays and reagents useful for identifying the presence of a disease or condition, or the propensity to develop a disease or condition, or the severity of a disease or condition wherein the disease or condition is characterized by being a bone pathology such as a cranial abnormality associated with fused sutures.

Hence, the present invention contemplates a method of diagnosing or predicting the development of a bone pathology in a subject, said method comprising isolating a sample from a potentially affected bone or bone tissue from the subject, said sample comprising genetic material or a protein or RNA encoded by the genetic material and determining the pattern of expression of the genetic material wherein up-regulation or down-regulation of expression of particular genetic material relative to a control is indicative of a bone pathology or risk of developing same.

The assays may, therefore, be genetic or protein based. Particularly useful diagnostic targets are listed above. A single target may be identified as being up- or down-regulated or an array of two or more may provide a profile which in itself provides an indication of the presence of a bone pathology or a risk of development of same.

A particularly preferred diagnostic assay is nucleic acid based such as but not limited to detecting mutations in DNA and levels and mutations of mRNA. Reference herein to DNA and mRNA means nucleic acid molecules associated with the biomarkers. In one embodiment, mutations in a biomarker or set of biomarkers are predictive of the potential for the development of a bone pathology such as but not limited to craniosynostosis. Reference herein to a sample from which a nucleic acid or protein based assay is conducted includes a biological sample such as serum, whole blood, plasma, mucus, tissue fluid, tissue extract, bone tissue biopsy or other source of genes or proteins associated with a bone pathology.

Expression levels of a gene can be altered by changes in the transcription of the gene product (i.e. transcription of mRNA) and/or by changes in translation of the gene product (i.e. translation of the protein) and/or by post-translation modification(s) (e.g. protein folding, glycosylation, etc.). Expression levels may also be affected by mutation levels in the gene. Thus preferred assays of the present invention include assaying for level of transcribed mRNA, level of translated protein, activity or translated protein and/or mutations including polymorphisms in DNA or mRNA.

For example, changes in expression level can be detected by measuring changes in mRNA and/or a nucleic acid derived from the mRNA (e.g. reverse-transcribed cDNA, etc.). In order to measure target gene expression level, it is desirable to provide a nucleic acid sample for such analysis. In preferred embodiments, the nucleic acid is found in or derived from a biological sample. The term “biological sample”, as used herein, refers to a sample obtained from an organism or from components (e.g. cells) of an organism. The sample may be of any biological tissue or fluid. Biological samples may also include organs or sections of tissues such as frozen sections taken for histological purposes. Generally, however, a bone sample may be taken or, in the case of craniosynostosis, the sample is from a calvarial suture.

The “control” is generally the expression pattern of genes in unfused versus fused calvarial sutures.

The nucleic acid (e.g. mRNA nucleic acid derived from mRNA) is, in certain preferred embodiments, isolated from the sample according to any of a number of methods well know to those skilled in the art. Methods of isolating mRNA are well known to those of skill in the art. For example, methods of isolation and purification of nucleic acids are described in detail in Tijssen, Ed, Chapter 3 of Laboratory Techniques in Biochemistry and Molecular Biology Hybridization with Nucleic Acid Probes, Part I, Theory and Nucleic Acid Preparation, Elsevier, N.Y. and Tijssen, Ed.

In a preferred embodiment, the “total” nucleic acid is isolated from a given sample using, for example, an acid guanidinium-phenol-chloroform extraction method and polyA+mRNA is isolated by oligo dT column chromatography or by using (dT)n magnetic beads (see Sambrook et al, Molecular Cloning: A Laboratory Manual (2^{nd} ed.) 1-3:1989 or Ausubel et al, Current Protocols in Molecular Biology, F, Greene Publishing and Wiley-Interscience, New York, 1987).

Frequently, it is desirable to amplify the nucleic acid sample prior to assaying for expression level. Methods of amplifying nucleic acids are well known to those of skill in the art and include, but are not limited to polymerase chain reaction (PCR, e.g. Innis et al, PCR Protocols, A Guide to Methods and Application. Academic Press, Inc. San Diego, 1990), ligase chain reaction (LCR) (see Wu and Wallace, Genomics 4:560, 1989; Landegren et al, Science 241:1077, 1988 and Barringer et al, Gene 89:117, 1990), transcription amplification (Kwoh et al, Proc. Natl. Acad. Sci. USA 86:1173, 1989), self-sustained sequence replication (Guatelli et al, Proc. Nat. Acad. Sci. USA 87:1874, 1990), dot PCR, and linker adapter PCR.

Using the known sequence of a target gene, detecting and/or quantifying the target gene transcript(s) can be routinely accomplished using nucleic acid hybridization techniques (see, Sambrook et al, 1989 supra). For example, one method for evaluating the presence, absence, or quantity of target gene reverse-transcribed cDNA involves a “Southern Blot”. In a Southern Blot, the DNA (e.g. reverse-transcribed target mRNA), typically fragmented and separated on an electrophoretic gel, is hybridized to a probe specific for the target gene. Comparison of the intensity of the hybridization signal from the target gene probe with a “control” probe (e.g. a probe for a “housekeeping gene”) provides an estimate of the relative expression level of the target nucleic acid.

Alternatively, the target gene mRNA can be directly quantified in a Northern blot. In brief, the mRNA is isolated from a given cell sample using, for example, an acid guanidinium-phenol-chloroform extraction method. The mRNA is then electrophoresed to separate the RNA species and the mRNA is transferred from the gel to a nitrocellulose membrane. As with the Southern blots, labeled probes are used to identify and/or quantify the target gene mRNA. Appropriate controls (e.g. probes to housekeeping genes) provide a reference for evaluating relative expression level.

An alternative means for determining the target gene expression level is in situ hybridization. In situ hybridization assays are well known (e.g. Angerer, Meth. Enzymol 152:649, 1987). Generally, in situ hybridization comprises the following major steps: (1) fixation of tissue or biological structure to be analyzed; (2) prehybridization treatment of the biological structure to increase accessibility of target DNA or RNA, and to reduce non-specific binding; (3) hybridization of the mixture of nucleic acids to the nucleic acid in the biological structure or tissue; (4) post-hybridization washes to remove nucleic acid fragments not bound in the hybridization; and (5) detection of the hybridized nucleic acid fragments. The reagent used in each of these steps and the conditions of use vary depending on the particular application.

In another embodiment, amplification-based assays can be used to measure target gene expression (transcription) level. In such amplification-based assays, the target nucleic acid sequences act as template(s) in amplification reaction(s) (e.g. Polymerase Chain Reaction (PCR) or reverse-transcription PCR(RT-PCR)). In a quantitative amplification, the amount of amplification product will be proportional to the amount of template in the original sample. Comparison to appropriate (e.g. healthy tissue or cells unexposed to the test agent) controls provides a measure of the target gene transcript level.

The present invention extends to array-based hybridization formats. Arrays are a multiplicity of different “probe” or “target” nucleic acids (or other compounds) attached to one or more surfaces (e.g. solid, membrane or gel). In a preferred embodiment, the multiplicity of nucleic acids (or other moieties) is attached to a single contiguous surface or to a multiplicity of surfaces juxtaposed to each other.

In an array format a large number of different hybridization reactions can be run essentially “in parallel”. This provides rapid, essentially simultaneous, evaluation of a number of hybridizations in a single “experiment”. Methods of performing hybridization reactions in array based formats are well known to those of skill in the art (see Pastinen, Genome Res. 7:606-6145, 1997; Jackson, Nature Biotechnology 14:1685, 1996; Chee, Science 274:610, 1995; WO 96/17958; Pinkel et al, Nature Genetics 20:207-211, 1998).

Arrays, particularly nucleic acid arrays can be produced according to a wide variety of methods well known to those of skill in the art. For example, in a simple embodiment, “low density” arrays can simply be produced by spotting (e.g. by hand using a pipette) different nucleic acids at different locations on a solid support (e.g. a glass surface, a membrane, etc.).

This simple spotting approach has been automated to produce high density spotted arrays (see U.S. Pat. No. 5,807,522). This patent describes the use of an automated system that taps a microcapillary against a surface to deposit a small volume of a biological sample. The process is repeated to generate high density arrays.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide arrays. Synthesis of high density arrays is also described in U.S. Pat. Nos. 5,744,305; 5,800,992 and 5,445,934.

In addition to, or in alternative to, the detection of target gene nucleic acid expression level(s), alterations in expression of a target gene can be detected and/or quantified by detecting and/or quantifying the amount and/or activity of a translated target gene encoded polypeptide.

The polypeptide(s) encoded by a target gene can be detected and quantified by any of a number of methods well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, Western blotting, and the like.

In one embodiment, the target gene expression product (e.g. proteins) are detected/quantified in an electrophoretic protein separation (e.g. a 1- or 2-dimensional electrophoresis). Means of detecting proteins using electrophoretic techniques are well known to those of skill in the art (see Scope, Protein Purification, Springer-Verlag, NY, 1982; Duetscher, Methods in Enzymology Vol. 182: Guide to Protein Purification, Academic Press, Inc. NY, 1990).

In another embodiment, Western blot (immunoblot) analysis is used to detect and quantify the presence of polypeptide(s) of the subject invention in the sample. This technique generally comprises separating sample proteins by gel electrophoresis on the basis of molecular weight, transferring the separated proteins to a suitable solid support (such as a nitrocellulose filter, a nylon filter, or derivatized nylon filter), and incubating the sample with the antibodies that specifically bind the target polypeptide(s).

Hence, the present invention extends to antibodies to target polypeptides. Polyclonal antibodies may conveniently be used, however, the use of monoclonal antibodies in an immunoassay or for capture is particularly preferred because of the ability to produce them in large quantities and the homogeneity of the product. The preparation of hybridoma cell lines for monoclonal antibody production is derived by fusing an immortal cell line and lymphocytes sensitized against the immunogenic preparation (i.e. comprising 35-LM polypeptide) or can be done by techniques which are well known to those who are skilled in the art. (See, for example, Douillard and Hoffman, Basic Facts about Hybridomas, in Compendium of Immunology Vol. II, ed. by Schwartz, 1981; Kohler and Milstein, Nature 256: 495-499, 1975; Kohler and Milstein, European Journal of Immunology 6: 511-519, 1976). Single chain antibodies or transgenic mice expressing humanized antibodies or other recognition proteins may also be used. Useful proteins in this regard include diabodies, peptide mimetics and antibody fragments such as scFv fragments and Fab fragments.

The present invention further provides therefore the application of biochemical techniques to render an antibody derived from one animal or avian creature substantially non-immunogenic in another animal or avian creature of the same or different species. The biochemical process is referred to herein as “de-immunization”. Reference herein to “de-immunization” includes processes such as complementary determinant region (CDR) grafting, “reshaping” with respect to a framework region of an immuno-interactive molecule and variable (v) region mutation, all aimed at reducing the immunogenicity of an immuno-interactive molecule in a particular host (eg. a human subject). In the present case, the preferred antibody is a monoclonal antibody, derived from one animal or avian creature and which exhibits reduced immunogenicity in another animal or avian creature from the same or different species such as but not limited to humans if used in a human form therapeutic or imaging purposes.

The present invention extends to antibodies or their antigen binding fragments. Antibodies may be polyclonal or monoclonal.

Polyclonal antibodies to a target polypeptide can be prepared using methods well-known to those of skill in the art (see, for example, Green et al, Immunochemical Protocols (Manson ed):1-5, 1992; Williams et al, DNA Cloning 2: Expression Systems, 2^{nd} Ed., Oxford University Press 1995). Although polyclonal antibodies are typically raised in animals such as rats, mice, rabbits, goats, or sheep, a target polypeptide antibody of the present invention may also be derived from a subhuman primate antibody. General techniques for raising diagnostically and therapeutically useful antibodies in baboons may be found, for example, in Goldenberg et al, International Patent Publication No. WO 91/11465, 1991 and in Losman et al, Int. J. Cancer 46:310, 1990.

The antibody should comprise at least a variable region domain. The variable region domain may be of any size or amino acid composition and will generally comprise at least one hypervariable amino acid sequence responsible for antigen binding embedded in a framework sequence. In general terms the variable (V) region domain may be any suitable arrangement of immunoglobulin heavy (V_{H}) and/or light (V_{L}) chain variable domains. Thus, for example, the V region domain may be monomeric and be a V_{H} or V_{L} domain where these are capable of independently binding antigen with acceptable affinity. Alternatively the V region domain may be dimeric and contain V_{H}-V_{H}, V_{H}-V_{L}, or V_{L}-V_{L}, dimers in which the V_{H} and V_{L} chains are non-covalently associated (abbreviated hereinafter as Fᵥ). Where desired, however, the chains may be covalently coupled either directly, for example via a disulphide bond between the two variable domains, or through a linker, for example a peptide linker, to form a single chain domain (abbreviated herein after as scFᵥ).

The variable region domain may be any naturally occurring variable domain or an engineered version thereof. By engineered version is meant a variable region domain that has been created using recombinant DNA engineering techniques. Such engineered versions include those created for example from natural antibody variable regions by insertions, deletions or changes in or to the amino acid sequences of the natural antibodies. Particular examples of this type include those engineered variable region domains containing at least one CDR and optionally one or more framework amino acids from antibody and the remainder of the variable region domain from a second antibody.

The variable region domain may be covalently attached at a C-terminal amino acid to at least one other antibody domain or a fragment thereof. Thus, for example, where a V_{H} domain is present in the variable region domain this may be linked to an immunoglobulin C_{H}1 domain or a fragment thereof. Similarly, a V_{L} domain may be linked to a C_{K} domain or a fragment thereof. In this way for example, the antibody may be a Fab fragment wherein the antigen binding domain contains associated V_{H} and V_{L} domains covalently linked at their C-termini to a C_{H}1 and C_{K} domain respectively. The C_{H}1 domain may be extended with further amino acids, for example to provide a hinge region domain as found in a Fab fragment, or to provide further domains, such as antibody CH2 and CH3 domains.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA or antibody-producing cells (see, for example, Larrick et al, Methods: A Companion to Methods in Enzymology 2:106, 1991; Courtneay-Luck, Monoclonal Antibodies: Production, Engineering and Clinical Application, Ritter et al (eds), Cambridge University Press:166, 1995 and Ward et al, Monoclonal Antibodies: Principles and Applications Birch et al, Wiley-Liss, Inc.:137, 1995.

Antibodies for use in the subject invention are preferably monoclonal (prepared by conventional immunization and cell fusion procedures) or in the case of fragments, derived therefrom using any suitable standard chemical such as reduction or enzymatic cleavage and/or digestion techniques, for example by treatment with pepsin. More specifically, monoclonal anti-TGF-beta binding-protein antibodies can be generated utilizing a variety of techniques. Rodent monoclonal antibodies to specific antigens may be obtained by methods known to those skilled in the art (see, for example, Kohler et al, 1975 supra and Coligan et al, Current Protocols in Immunology 1, John Wiley & Sons 1991; Picksley et al, DNA Cloning 2: Expression Systems, 2^{nd} Edition, Glover et al (eds), page 93 Oxford University Press, 1995).

Briefly, monoclonal antibodies can be obtained by injecting mice with a composition comprising a target gene product, verifying the presence of antibody production by removing a serum sample, removing the spleen to obtain B-lymphocytes, fusing the B-lymphocytes with myeloma cells to produce hybridomas, cloning the hybridomas, selecting positive clones which produce antibodies to the antigen, culturing the clones that produce antibodies to the antigen, and isolating the antibodies from the hybridoma cultures.

In addition, an anti-target polypeptide antibody of the present invention may be derived from a human monoclonal antibody. Human monoclonal antibodies are obtained from transgenic mice that have been engineered to produce specific human antibodies in response to antigenic challenge. In this technique, elements of the human heavy and light chain locus are introduced into strains of mice derived from embryonic stem cell lines that contain targeted disruptions of the endogenous heavy chain and light chain loci. The transgenic mice can synthesize human antibodies specific for human antigens and the mice can be used to produce human antibody-secreting hybridomas. Methods for obtaining human antibodies from transgenic mice are described, for example, by Green et al, Nature Genet. 7:1994, Lonberg et al, Nature 368:856, 1994 and Taylor et al, Int. Immun. 6:579, 1994).

Monoclonal antibodies can be isolated and purified from hybridoma cultures by a variety of well-established techniques. Such isolation techniques include affinity chromatography with Protein-A Sepharose, size-exclusion chromatography and ion-exchange chromatography (see for example, Baines et al, Methods in Molecular Biology 10:79-104, 1992).

For particular uses, it may be desirable to prepare fragments of anti-target polypeptide antibodies. Such antibody fragments can be obtained by pepsin or papain digestion of whole antibodies by conventional methods. As an illustration, antibody fragments can be produced by enzymatic cleavage of antibodies with pepsin to provide a 5 S fragment denoted F(ab′)₂. This fragment can be further cleaved using a thiol reducing agent to produce 3.5 S Fab′ monovalent fragments. Optionally, the cleavage reaction can be performed using a blocking group for the sulfhydryl groups that result from cleavage of disulfide linkages. As an alternative, an enzymatic cleavage using pepsin produces two monovalent Fab fragments and an Fc fragment directly. These methods are described, for example, by Goldenberg U.S. Pat. No. 4,331,647; Nisonoff et al, Arch Biochem. Biophys. 89:230, 1960; Porter, Biochem J. 73:119, 1959; Edelman et al, Enzymology 1:422, 1967).

Other methods of cleaving antibodies, such as separation of heavy chains to form monovalent light-heavy chain fragments, further cleavage of fragments, or other enzymatic, chemical or genetic techniques may also be used, so long as the fragments bind to the antigen that is recognized by the intact antibody.

Alternatively, the antibody may be a recombinant or engineered antibody obtained by the use of recombinant DNA techniques involving the manipulation and re-expression of DNA encoding antibody variable and/or constant regions. Such DNA is known and/or is readily available from DNA libraries including for example phage-antibody libraries (see Chiswell and McCafferty, J. Tibtech 10:80-84, 1992) or where desired can be synthesized. Standard molecular biology and/or chemistry procedures may be used to sequence and manipulate the DNA, for example, to introduce codons to create cysteine residues, to modify, add or delete other amino acids or domains as desired.

One or more replicable expression vectors containing the DNA encoding a variable and/or constant region may be prepared and used to transform an appropriate cell line, e.g. a non-producing myeloma cell line, such as bacterial (e.g. E. coli) in which production of the antibody will occur. In order to obtain efficient transcription and translation, the DNA sequence in each vector should include appropriate regulatory sequences, particularly a promoter and leader sequence operably linked to a variable domain sequence. Particular methods for producing antibodies in this way are generally well known and routinely used. For example, basic molecular biology procedures are described by Maniatis et al, Molecular Cloning, Cold Spring Harbor Laboratory, New York, 1989; DNA sequencing can be performed as described in Sanger et al, Proc Natl. Acad Sci USA 74:5463, 1977 and the Amersham International plc sequencing handbook; site directed mutagenesis can be carried out according to the method of Kramer et al, Nucleic Acids Res. 12:9441, 1984; the Anglian Biotechnology Ltd Handbook, Kunkel, Proc. Natl. Acad. Sci. USA 82:488-492, 1985; Kunkel et al, Methods in Enzymol. 154:367-382, 1987). Additionally, numerous publications detail techniques suitable for the preparation of antibodies by manipulation of DNA, creation of expression vectors, and transformation of appropriate cells, for example as reviewed by Mountain A and Adair J R in Biotechnology and Genetic Engineering Reviews (ed. Tombs, M. 10, Chapter 1) Intercept, Andover, UK, 1992 and in International Patent Specification No. WO 91/09967.

In certain embodiments, the antibody according to the present invention may have one or more effector or reporter molecules attached to it and the subject invention extends to such modified proteins. A reporter molecule may be a detectable moiety or label such as an enzyme, or other reporter molecule, including a dye, radionuclide, luminescent group, fluorescent group, or biotin, or the like. The target polypeptide specific immunoglobulin or fragment thereof may be radiolabeled for diagnostic or therapeutic applications. Techniques for radiolabeling of antibodies are known in the art, see Adams, In Vivo 12:11-21, 1998; Hiltunen, Acta Oncol. 32:931-939, 1993. The effector or receptor molecules may be attached to the antibody through any available amino acid side-chain, terminal acid, or where present, carbohydrate functional group located in the antibody, provided that the attachment or the attachment process does not adversely affect the binding properties and the usefulness of the molecule. Particular functional groups include, for example, any free amino, imino, thiol, hydroxyl, carboxyl or aldehyde group. Attachment of the antibody and the effector and/or reporter molecule(s) may be achieved via such groups and an appropriate functional group in the effector or reporter molecules. The linkage may be direct or indirect through spacing or bridging groups.

The antibodies of the present invention may be used both therapeutically to inhibit or target a protein or may be used diagnostically to screen for levels of target proteins.

In terms of diagnostic assays as indicated above, the gene product or antibodies or nucleic acid molecules described above, may be labeled with a variety of compounds, including for example, fluorescent molecules, toxins and radionuclides. Representative examples of fluorescent molecules include fluorescin, Phycobili proteins such as phycoerythrin, rhodamine, Texas red and luciferase. Representative examples of toxins include ricin, abrin, diphtheria toxin, cholera toxin, gelonin, pokeweed antiviral protein, tritin, Shigella toxin, and Pseudomonas exotosin A. Representative examples of radionuclides include Cu-64, Ga-67, GA-68, Zr-89, Ru-97, Tc-99m, Rh-105, Pd-109, In-111, I-123, I-125, I-131, Re-186, Re-188, Au-198, Au-199, Pb-203, At-211, Pb-212 and Bi-212. In addition, the antibodies described above may also be labeled or conjugated to one partner of a ligand binding pair. Representative examples include avidin-biotin, streptavidin-biotin, and riboflavin-riboflavin binding protein.

Methods for conjugating or labeling the molecules described herein with the representative labels set forth above may be readily accomplished by one of ordinary skill in the art (see Trichothecene Antibody Conjugate, U.S. Pat. No. 4,744,981; Antibody Conjugate, U.S. Pat. No. 5,106,951; Fluorogenic Materials and Labeling Techniques U.S. Pat. No. 4,018,884; Metal Radionuclide Labeled Proteins for Diagnosis and Therapy U.S. Pat. No. 4,897,255; and Metal Radionuclide Chelating Compounds for Improved Chelation Kinetics U.S. Pat. No. 4,988,496; see also Inman, Methods In Enzymology 34:30, 1974; Wilchek and Bayer, Anal. Biochem. 171:1-32, 1988).

Diagnostic and therapeutic kits and compositions also form part of the present invention. Such kits may comprise diagnostic or therapeutic agents, singularly or in combination with other agents.

Hence, another aspect of the present invention is directed to the use of an agent which up-regulates or down-regulates a gene listed in Table 2 or 3 or 4 in the manufacture of a medicament or diagnostic agent for a bone pathology in a subject.

The present invention is further described by the following non-limiting examples.

### Example 1

### Identification of Bone Pathology-Associated Biomarkers

RNA was extracted from calvarial sutures from 5 patients (males ages 3-7 months) with craniosynostosis. Suture tissue was obtained from both unfused and fused sutures from the patients. Gene expression within each tissue was analyzed using Affymetrix U133A2.0 GeneChips. Expression patterns were compared between all unfused sutures and all fused sutures. Bioinformatics was applied to the microarray data to identify genes with significant differential expression. Combining all sutures together ensured that the genes identified are expressed in each of the coronal, sagittal and lambdoid sutures and, therefore, do not have a suture-specific effect.

11 genes have been analyzed by quantitative realtime RT-PCR to validate the microarray data, using RNA from the initial sample set and four additional patients. 89% correlation was achieved, indicating the microarray data are robust.

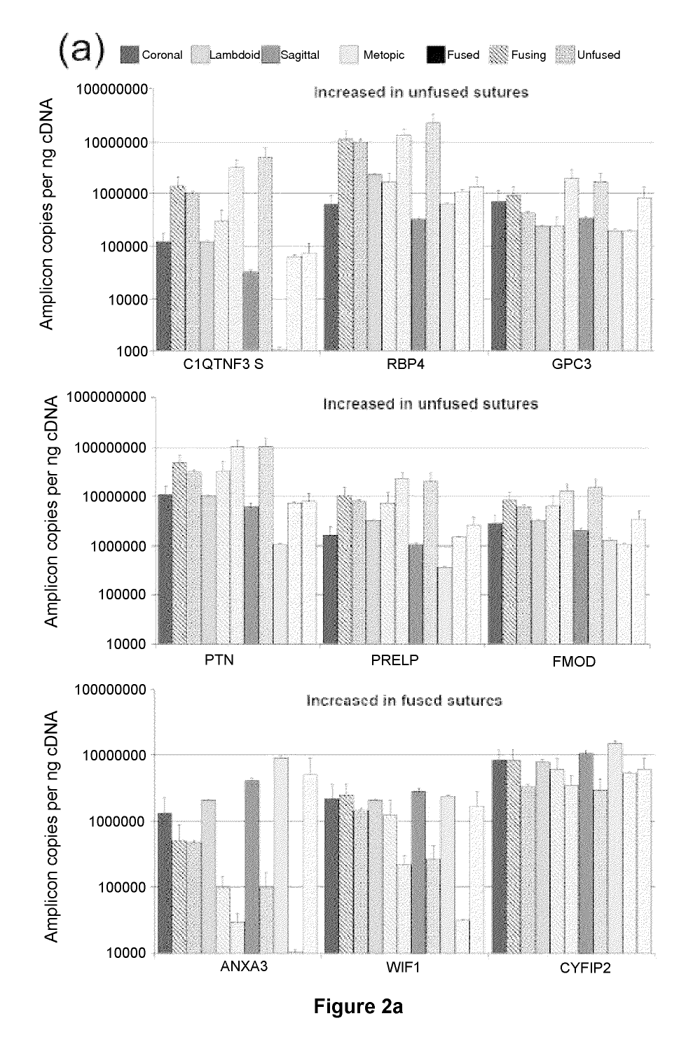

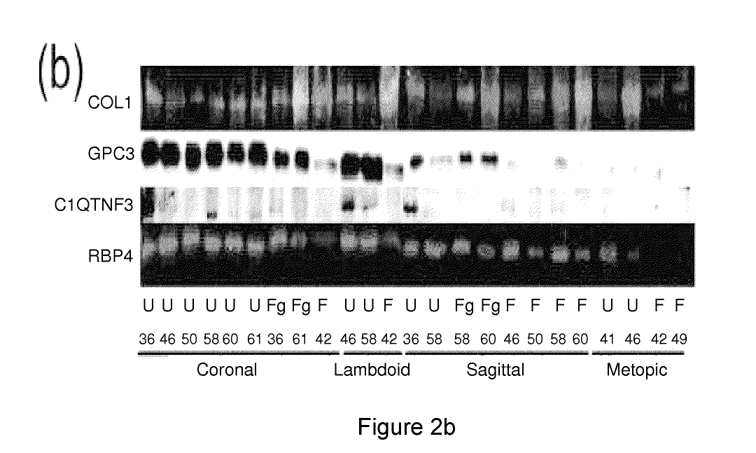

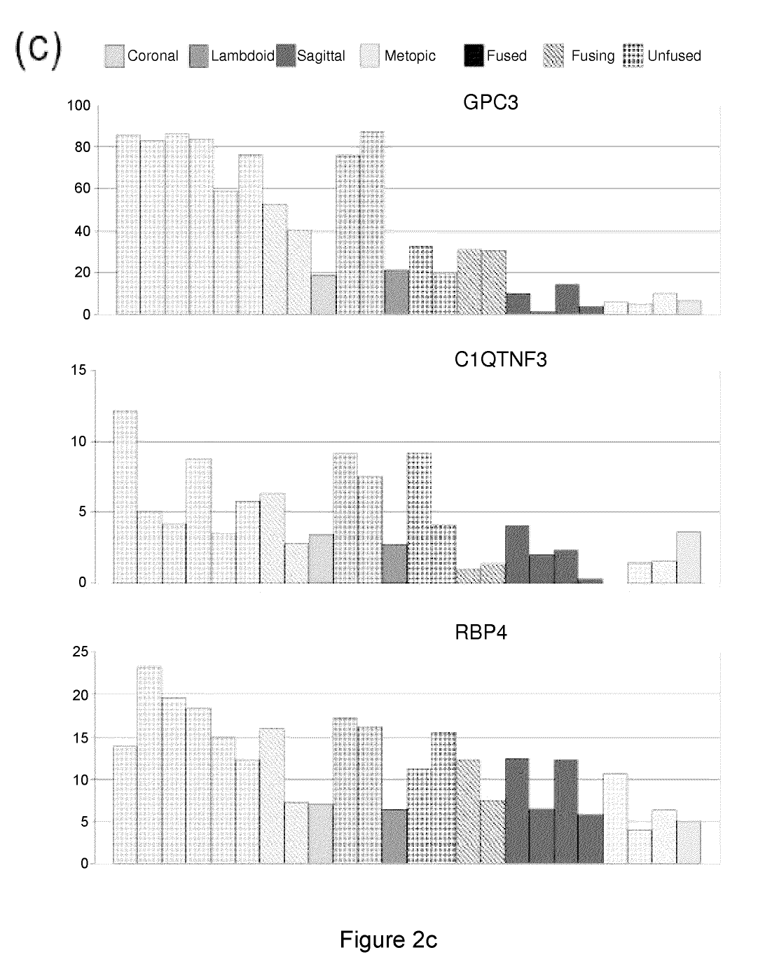

Biomarker expression has been analyzed in cultured primary cells over several passages. Under unmodified culturing conditions, the biomarker expression exhibited by the tissue does not correlate in the cells. This indicates that culturing conditions may need modification to regain correct expression of the biomarkers. These markers are, therefore, useful tools in ensuring the cells are maintaining in vivo expression. FIG. 2 provides mRNA and protein validation of differential expression identified by microanalysis. The expression patterns of RBP4 and GPC3 are shown in FIG. 3.

A summary of functions of the key genes identified follow:

Upregulated in unfused: the following genes are predicted to be pivotal in maintaining suture patency or in controlling early osteoblast differentiation.

RBP4 is a binder and carrier of retinol (vitamin A). All trans-retinoic acid (RA) is a metabolite of retinol and is a known craniosynostosis causing teratogen (Gardner et al, Int. J. Epidemiol 27(1):64-67, 1998; Yip et al, Teratology 21(1):29-38, 1980). Studies show RA increases differentiation of osteoblasts, decreases proliferation and induces bone nodule formation in vitro (Song et al, J. Cell Physiol. 202(1):255-262, 2005; Cowan et al, Tissue Eng. 11(3-4):645-658, 2005). One potential function of RBP4 in suture mesenchyme may be to sequester retinol. Once its expression is downregulated (during the fusing stage) retinol is released and converted to RA, which then stimulates osteoblast differentiation and bone formation. An inhibitor of RBP4 may, therefore, promote osteogenesis, while delivery of the secreted protein will maintain proliferation of early stage osteoblasts and limit terminal differentiation.

C1QTNF3 was initially identified in a chondrocyte cell line after treatment with TGF-[3]. Embryonic expression analysis in mice show a high level of expression in prechondrocytic mesenchymal cells, but it is undetectable in mature chondrocytes (Maeda et al, J. Biol. Chem. 276(5):3628-3634, 2001). Our work is the first to identify C1QTNF3 in calvarial suture preosteoblastic mesenchyme and suggests a possible function in regulating mesenchymal condensations during skeletal development.

GPC3 is a cell surface heparan sulphate proteoglycan. Loss of GPC3 causes Simpson-Golabi Behmel syndrome, which is characterised by pre- and post-natal overgrowth, cleft palate, short broad nose, prognathism, widened nasal bridge and disproportionably large head. Double mutant mice for BMP4 and GPC3 have increased phenotype, suggesting GPC3 is involved in BMP signaling. Ectopic GPC3 expression decreases BMP4 expression and blocks BMP7 activity (Midorikawa et al, Int. J. Cancer 103(4):455-465, 2003; Paine-Saunders et al, Dev. Biol. 225(1):179-187, 2000), suggesting GPC3 acts to limit BMP induced osteoblast differentiation. GPC3 deficient mice also present with polydactyl, a common phenotype seen in patients with craniosynostosis syndromes. GPC3 has also been shown to bind FGF2 and overexpression of GPC3 suppressed FGF2-induced cell proliferation in hepatocytes (Midorikawa et al, 2003 supra). This suggests that GPC3 also interacts in FGF signaling on osteoprogenitors, as FGFR mutations are the common cause of multiple craniosynostosis syndromes. Furthermore, GPC3 has been shown to suppress non-canonical Wnt signaling and activate canonical Wnt/β-Catenin signaling and in doing so regulates cell proliferation (Song et al, J. Biol. Chem. 280(3):2116-2125, 2005; De Cat et al, J. Cell Biol. 163(3):625-635, 2003). There are multiple avenues through which GPC3 may control cell growth within osteogenic mesenchyme, but inhibition leads to increased growth, while inducing GPC3 suppresses signaling pathways involving FGFs, BMPs and non-canonical Wnts.

MFAP4 is a putative ECM protein involved in cell adhesion or cell to cell interaction. Deletion of MFAP4 causes Smith-Magenis syndrome, clinical features of which are brachycephaly, midface hypoplasia, prognathism and growth retardation (Zhao et al, Hum. Mol. Genet. 4(4):589-597, 1995). The skull malformation suggests MFAP4 may be vial component of suture mesenchyme. Bovine Mfap4 has been identified as a collagen binder and may aid in ECM organization (Lausen et al, Biol. Chem. 274(45):32234-32240, 1999).

FMOD is a small leucine-rich proteoglycan (SLRP) and is involved in ECM assembly. It competes for binding with TGFβ1, 2 and 3 and may sequester it in the ECM (Hildebrand, 1994). It is a regulator for collagen fibrillogenesis, and its RNA expression is upregulated just before the onset of mineralization. It bind collagen, retarding the rate of fibril formation leading to thinner fibrils. It is been identified as being upregulated by BMP2 dependent differentiation of C2C12 premyoblasts into the osteogenic lineage. It may regulate cellular growth or migration.

OGN is a small leucine-rich keratan sulphate proteoglycan which induces ectopic bone formation in conjunction with transforming growth factor beta. It is thought that osteoglycin may regulate cellular growth as its transcription is up regulated by growth factors and tumor suppressor protein p53. Furthermore, it has been shown to inhibit multinucleated cell formation and subsequently limit osteoclast formation and activity. Mice deficient in OGN also have increased collagen fibril diameter, indicating a role in collagen fibrillogenesis.

PRELP is a heparin-binding small leucine-rich proteoglycan (SLRP) in connective tissue extracellular matrix. PRELP binds the basement membrane heparan sulfate proteoglycan perlecan. PRELP binds collagen type I and type II. It is that PRELP functions as a molecule anchoring basement membranes to the underlying connective tissue.

Upregulated in fused: the following genes are predicted to play roles in suture fusion.

WIF1 is an antagonist of Wnt signaling and has recently been shown to have strong expression in late phase differentiation of C2C12 and MC3T3E-1 preosteoblast cell lines. Continuous activation of Wnt signaling reduces osteoblast differentiation, thus WIF1 may be required for controlling osteoblast maturation (Vaes et al, Bone 36(5):803-811, 2005).

ANXA3 is a member of the calcium-dependent phospholipid-binding protein family. Limited information is known about ANXA3, however, general AnxA expression has been identified to be significantly increased during progression of osteoarthritis. S100A proteins have been shown to interact with AnxA5 and AnxA6 and as a number of S100A proteins are also upregulated with ANXA3, they may interact with ANXA3 as well. Retinol can bind AnxA6 and inhibition of ion channel activity of AnxAs has been shown to reverse the apoptotic effects of RA (Balcerzak et al, FEBS Lett. 580:3065-3069, 2006). RA has also been shown to stimulate cell differentiation and expression of AnxAs in avian growth plate chondrocytes, suggesting a link between ANXA3 and retinoic acid induced osteogenesis.

CASP1 has been shown to induce cell apoptosis and may function in various developmental stages. This gene was identified by its ability to proteolytically cleave and activate the inactive precursor of interleukin-1, a cytokine involved in the processes such as inflammation, septic shock, and wound healing.

SHOX2 is a member of the homeo box family and has a C-terminal 14-amino acid residue motif characteristic for craniofacially expressed homeodomain proteins. Limited embryo expression analysis identified SHOX2 in condensing CART1 laginous mesenchyme in the nose and palate In the fore limb bud, transcripts were restricted to undifferentiated mesenchyme condensing around the developing bone (Rudiger et al, Proc. Natl. Acad. Sci. USA 95(5):2406-2411, 1998).

CYFIP2, cytoplasmic FMR1 (fragile X mental retardation 1) interacting protein, is an actin regulatory protein and a mediator of p53-dependent apoptosis. It increases fibronectin-mediated binding in Jurkat and CD4(+) cells (Mayne et al, Eur J. Immunol. 34:1217-27, 2004). These studies suggest that overabundance of CYFIP2 protein facilitates increased adhesion properties of T cells from MS patients. CYFIP2 may therefore be involved in an immune-like response during suture fusion.

Genes showing at least a 2-fold increase in expression in unfused sutures and fused sutures are shown in Tables 2 and 3, respectively.

### Example 2

### Biomarker Identification

Using the methodology of Example 1, a particular list of biomarkers was identified. The biomarkers are shown in Table 4.

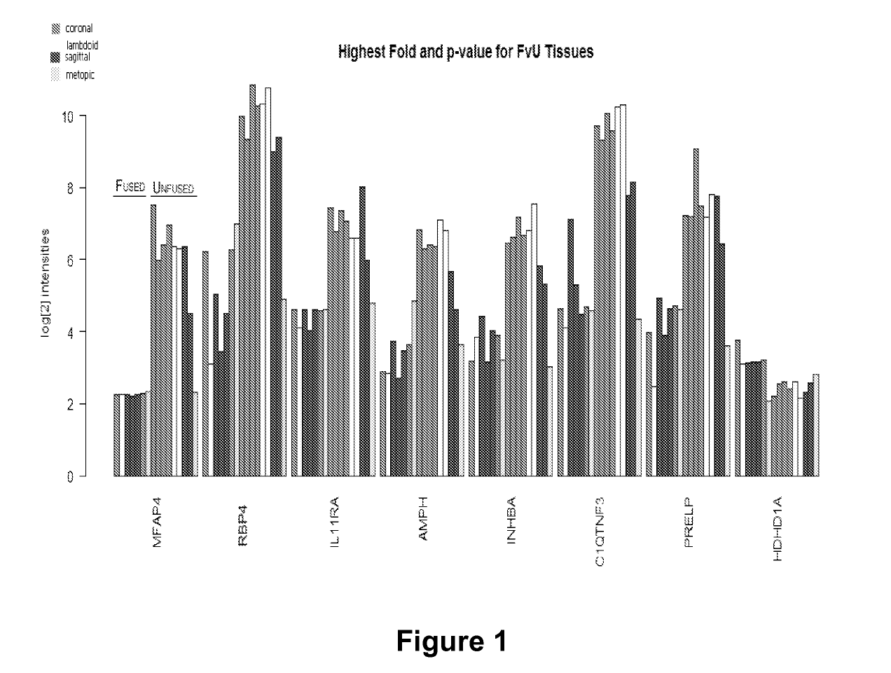

FIG. 1 shows the level of expression of one particular biomarker, RBP4 in fused and unfused sutures.

Table 5 shows the expression of several preferred biomarkers in human bone cancer cell lines.

TABLE 2
Up-regulated in Unfused sutures and bone cell growth i.e. maintain patency:

| | Fold | | | |
|---|---|---|---|---|
| ID | Change | P. Value | Gene | Name |
| 212713_at | 16.5018 | 0.000831 | MFAP4 | microfibrillar-associated protein 4 |
| 219140_s_at | 37.3812 | 0.002791 | RBP4 | retinol binding protein 4 (plasma) KIAA1922 |
| 204773_at | 5.9024 | 0.002791 | IL11RA | interleukin 11 receptor, alpha |
| 205257_s_at | 8.2692 | 0.005983 | AMPH | amphiphysin (Stiff-Man syndrome with breast cancer 128 kDa autoantigen) |
| 210511_s_at | 6.9377 | 0.005983 | INHBA | inhibin, beta A (activin A, activin AB alpha polypeptide) |
| 220988_s_at | 20.2546 | 0.007101 | C1QTNF3 | C1q and tumor necrosis factor related protein 3 |
| 204223_at | 10.6493 | 0.008592 | PRELP | proline arginine-rich end leucine-rich repeat protein |
| 202994_s_at | 9.9691 | 0.02418 | FBLN1 | fibulin 1 |
| 213004_at | 6.6661 | 0.02418 | ANGPTL2 | angiopoietin-like 2 |
| 217161_x_at | 6.1586 | 0.02418 | AGC1 | aggrecan 1 (chondroitin sulfate proteoglycan 1, large aggregating proteoglycan, antigen |
| | | | | identified by monoclonal antibody A0122) |
| 213001_at | 5.7696 | 0.02418 | ANGPTL2 | angiopoietin-like 2 |
| 202709_at | 5.4068 | 0.02418 | FMOD | fibromodulin |
| 205591_at | 2.5504 | 0.02418 | OLFM1 | olfactomedin 1 |
| 217967_s_at | 2.0372 | 0.02418 | C1orf24 | chromosome 1 open reading frame 24 |
| 202995_s_at | 12.8768 | 0.02547 | FBLN1 | fibulin 1 |
| 207692_s_at | 6.5867 | 0.02547 | AGC1 | aggrecan 1 (chondroitin sulfate proteoglycan 1, large aggregating proteoglycan, antigen |
| | | | | identified by monoclonal antibody A0122) |
| 205679_x_at | 5.8621 | 0.02547 | AGC1 | aggrecan 1 (chondroitin sulfate proteoglycan 1, large aggregating proteoglycan, antigen |
| | | | | identified by monoclonal antibody A0122) |
| 204964_s_at | 4.3992 | 0.02547 | SSPN | sarcospan (Kras oncogene-associated gene) |
| 201787_at | 6.3960 | 0.031356 | FBLN1 | fibulin 1 |
| 209466_x_at | 6.2533 | 0.031356 | PTN | pleiotrophin (heparin binding growth factor 8, neurite growth-promoting factor 1) |
| 205330_at | 3.7242 | 0.031356 | MN1 | meningioma (disrupted in balanced translocation) 1 |
| 211737_x_at | 5.5507 | 0.037262 | PTN | pleiotrophin (heparin binding growth factor 8, neurite growth-promoting factor 1) |
| 215271_at | 6.6681 | 0.037697 | TNN | tenascin N |
| 201983_s_at | 2.7105 | 0.037697 | EGFR | epidermal growth factor receptor (erythroblastic leukemia viral (v-erb-b) oncogene homolog, |
| | | | | avian) |
| 213217_at | 2.9611 | 0.038615 | ADCY2 | adenylate cyclase 2 (brain) |
| 212915_at | 3.7105 | 0.039056 | PDZRN3 | PDZ domain containing RING finger 3 |
| 213994_s_at | 4.7326 | 0.041097 | SPON1 | spondin 1, extracellular matrix protein |
| 209465_x_at | 4.1138 | 0.041097 | PTN | pleiotrophin (heparin binding growth factor 8, neurite growth-promoting factor 1) |
| 213993_at | 3.7365 | 0.041097 | SPON1 | spondin 1, extracellular matrix protein |
| 209220_at | 7.1306 | 0.049587 | GPC3 | glypican 3 |
| 205523_at | 7.1035 | 0.049587 | HAPLN1 | hyaluronan and proteoglycan link protein 1 |
| 219528_s_at | 5.6645 | 0.054791 | BCL11B | B-cell CLL/lymphoma 11B (zinc finger protein) |
| 219250_s_at | 5.1981 | 0.05481 | FLRT3 | fibronectin leucine rich transmembrane protein 3 |
| 219514_at | 8.9447 | 0.060506 | ANGPTL2 | angiopoietin-like 2 |
| 220994_s_at | 3.2406 | 0.063213 | STXBP6 | syntaxin binding protein 6 (amisyn) |
| 203083_at | 6.6221 | 0.06864 | THBS2 | thrombospondin 2 |
| 200906_s_at | 3.8483 | 0.070277 | KIAA0992 | NA |
| 215077_at | 6.3172 | 0.071678 | COL3A1 | collagen, type III, alpha 1 (Ehlers-Danlos syndrome type IV, autosomal dominant) |
| 214927_at | 3.2051 | 0.071678 | NA | NA |
| 217966_s_at | 2.4155 | 0.080315 | C1orf24 | chromosome 1 open reading frame 24 |
| 212958_x_at | 3.4234 | 0.08045 | PAM | peptidylglycine alpha-amidating monooxygenase |
| 202336_s_at | 3.3084 | 0.08045 | PAM | peptidylglycine alpha-amidating monooxygenase |
| 202245_at | 2.1081 | 0.08161 | LSS | lanosterol synthase (2,3-oxidosqualene-lanosterol cyclase) |
| 204320_at | 3.3349 | 0.082173 | COL11A1 | collagen, type XI, alpha 1 |
| 204529_s_at | 3.6382 | 0.088141 | TOX | NA |
| 202341_s_at | 2.9788 | 0.088141 | TRIM2 | tripartite motif-containing 2 |
| 221900_at | 11.4949 | 0.09203 | COL8A2 | collagen, type VIII, alpha 2 |
| 221541_at | 2.5496 | 0.09203 | CRISPLD2 | cysteine-rich secretory protein LCCL domain containing 2 |
| 221530_s_at | 5.9523 | 0.093217 | BHLHB3 | basic helix-loop-helix domain containing, class B, 3 |
| 209589_s_at | 2.3895 | 0.093217 | EPHB2 | EPH receptor B2 |
| 221563_at | 4.5284 | 0.094815 | DUSP10 | dual specificity phosphatase 10 |
| 37892_at | 3.6495 | 0.094815 | COL11A1 | collagen, type XI, alpha 1 |
| 213131_at | 3.0344 | 0.094815 | OLFM1 | olfactomedin 1 |
| 204141_at | 4.3522 | 0.094977 | TUBB2 | tubulin, beta 2 |
| 205933_at | 2.4872 | 0.094977 | SETBP1 | SET binding protein 1 |
| 205805_s_at | 2.2580 | 0.094977 | ROR1 | receptor tyrosine kinase-like orphan receptor 1 |
| 209909_s_at | 3.7615 | 0.096126 | TGFB2 | transforming growth factor, beta 2 |
| 207191_s_at | 3.7338 | 0.096126 | ISLR | immunoglobulin superfamily containing leucine-rich repeat |
| 201185_at | 2.3176 | 0.096126 | PRSS11 | protease, serine, 11 (IGF binding) |
| 204345_at | 2.2037 | 0.096126 | COL16A1 | collagen, type XVI, alpha 1 |
| 200872_at | 2.0132 | 0.096126 | S100A10 | S100 calcium binding protein A10 (annexin II ligand, calpactin I, light polypeptide (p11)) |
| 52651_at | 6.0367 | 0.099526 | COL8A2 | collagen, type VIII, alpha 2 |
| 203570_at | 2.9300 | 0.099526 | LOXL1 | lysyl oxidase-like 1 |
| 215945_s_at | 2.0100 | 0.099526 | TRIM2 | tripartite motif-containing 2 |
| 214981_at | 5.8716 | 0.101 | POSTN | periostin, osteoblast specific factor |
| 202998_s_at | 4.0304 | 0.101 | LOXL2 | lysyl oxidase-like 2 |
| 200953_s_at | 2.0429 | 0.10274 | CCND2 | cyclin D2 |
| 204963_at | 4.0633 | 0.108567 | SSPN | sarcospan (Kras oncogene-associated gene) |
| 219038_at | 4.4088 | 0.111333 | ZCWCC2 | zinc finger, CW type with coiled-coil domain 2 |
| 205422_s_at | 3.2048 | 0.111333 | ITGBL1 | integrin, beta-like 1 (with EGF-like repeat domains) |
| 219093_at | 2.0960 | 0.11503 | FLJ20701 | NA |
| 214620_x_at | 3.1198 | 0.118157 | PAM | peptidylglycine alpha-amidating monooxygenase |
| 201125_s_at | 2.0752 | 0.118821 | ITGB5 | integrin, beta 5 |
| 203417_at | 3.9290 | 0.120427 | MFAP2 | microfibrillar-associated protein 2 |
| 201615_x_at | 3.6820 | 0.120427 | CALD1 | caldesmon 1 |
| 204897_at | 2.5731 | 0.128279 | PTGER4 | prostaglandin E receptor 4 (subtype EP4) |
| 215506_s_at | 3.6814 | 0.135926 | DIRAS3 | DIRAS family, GTP-binding RAS-like 3 |
| 209561_at | 3.0859 | 0.135926 | THBS3 | thrombospondin 3 |
| 206510_at | 2.7201 | 0.135993 | SIX2 | sine oculis homeobox homolog 2 (Drosophila) |
| 212091_s_at | 3.0239 | 0.136213 | COL6A1 | collagen, type VI, alpha 1 |
| 219523_s_at | 2.5477 | 0.136318 | ODZ3 | odz, odd Oz/ten-m homolog 3 (Drosophila) |
| 201792_at | 2.2074 | 0.142169 | AEBP1 | AE binding protein 1 |
| 213428_s_at | 2.3280 | 0.14411 | COL6A1 | collagen, type VI, alpha 1 |
| 200907_s_at | 2.8637 | 0.145863 | KIAA0992 | NA |
| 201842_s_at | 5.0712 | 0.148454 | EFEMP1 | EGF-containing fibulin-like extracellular matrix protein 1 |
| 212554_at | 3.9508 | 0.154491 | CAP2 | CAP, adenylate cyclase-associated protein, 2 (yeast) |
| 205399_at | 2.5783 | 0.156697 | DCAMKL1 | doublecortin and CaM kinase-like 1 |
| 201124_at | 2.5136 | 0.156697 | ITGB5 | integrin, beta 5 |
| 205524_s_at | 8.7108 | 0.157867 | HAPLN1 | hyaluronan and proteoglycan link protein 1 |
| 218309_at | 2.6217 | 0.157867 | CaMKIINalpha | NA |
| 209436_at | 4.1194 | 0.159134 | SPON1 | spondin 1, extracellular matrix protein |
| 201465_s_at | 4.4081 | 0.160984 | JUN | v-jun sarcoma virus 17 oncogene homolog (avian) |
| 218638_s_at | 3.9345 | 0.16387 | SPON2 | spondin 2, extracellular matrix protein |
| 204471_at | 4.1460 | 0.165282 | GAP43 | growth associated protein 43 |
| 209692_at | 2.6475 | 0.165282 | EYA2 | eyes absent homolog 2 (Drosophila) |
| 219511_s_at | 2.1837 | 0.165282 | SNCAIP | synuclein, alpha interacting protein (synphilin) |
| 204037_at | 2.0603 | 0.165282 | EDG2 | endothelial differentiation, lysophosphatidic acid G-protein-coupled receptor, 2 |
| 215116_s_at | 6.2064 | 0.165825 | DNM1 | dynamin 1 |
| 220595_at | 4.8900 | 0.165825 | PDZRN4 | PDZ domain containing RING finger 4 |
| 218730_s_at | 5.7091 | 0.165976 | OGN | osteoglycin (osteoinductive factor, mimecan) |
| 219087_at | 2.4083 | 0.170985 | ASPN | asporin (LRR class 1) |
| 203636_at | 2.3726 | 0.170985 | MID1 | midline 1 (Opitz/BBB syndrome) |
| 214021_x_at | 2.0529 | 0.170985 | ITGB5 | integrin, beta 5 |
| 206114_at | 2.7478 | 0.173432 | EPHA4 | EPH receptor A4 |
| 206306_at | 5.1664 | 0.175566 | RYR3 | ryanodine receptor 3 |
| 215828_at | 2.0238 | 0.180193 | ATBF1 | AT-binding transcription factor 1 |
| 212732_at | 6.2244 | 0.183603 | MEG3 | maternally expressed 3 |
| 204753_s_at | 3.5411 | 0.209534 | HLF | hepatic leukemia factor |
| 209626_s_at | 2.5144 | 0.209534 | OSBPL3 | oxysterol binding protein-like 3 |
| 205226_at | 5.3494 | 0.210737 | PDGFRL | platelet-derived growth factor receptor-like |
| 222101_s_at | 2.1801 | 0.215541 | DCHS1 | dachsous 1 (Drosophila) |
| 202756_s_at | 3.0975 | 0.219174 | GPC1 | glypican 1 |
| 214464_at | 2.2422 | 0.219174 | CDC42BPA | CDC42 binding protein kinase alpha (DMPK-like) |
| 200636_s_at | 2.1157 | 0.219174 | PTPRF | protein tyrosine phosphatase, receptor type, F |
| 203638_s_at | 3.2069 | 0.219671 | FGFR2 | fibroblast growth factor receptor 2 (bacteria-expressed kinase, keratinocyte growth factor |
| | | | | receptor, craniofacial dysostosis 1, Crouzon syndrome, Pfeiffer syndrome, Jackson-Weiss |
| | | | | syndrome) |
| 40837_at | 3.0931 | 0.219671 | TLE2 | transducin-like enhancer of split 2 (E(sp1) homolog, Drosophila) |
| 201438_at | 2.0925 | 0.220059 | COL6A3 | collagen, type VI, alpha 3 |
| 200897_s_at | 2.4132 | 0.220064 | KIAA0992 | NA |
| 219427_at | 3.0206 | 0.225122 | FAT4 | FAT tumor suppressor homolog 4 (Drosophila) |
| 202827_s_at | 2.3869 | 0.226221 | MMP14 | matrix metalloproteinase 14 (membrane-inserted) |
| 203637_s_at | 2.1474 | 0.228907 | MID1 | midline 1 (Opitz/BBB syndrome) |
| 213519_s_at | 3.8440 | 0.230413 | LAMA2 | laminin, alpha 2 (merosin, congenital muscular dystrophy) |
| 200770_s_at | 2.0909 | 0.230413 | LAMC1 | laminin, gamma 1 (formerly LAMB2) |
| 221950_at | 2.7849 | 0.235217 | EMX2 | empty spiracles homolog 2 (Drosophila) |
| 205122_at | 3.2685 | 0.242931 | TMEFF1 | transmembrane protein with EGF-like and two follistatin-like domains 1 |
| 209633_at | 2.1835 | 0.242931 | PPP2R3A | protein phosphatase 2 (formerly 2A), regulatory subunit B″, alpha |
| 202747_s_at | 2.8957 | 0.243384 | ITM2A | integral membrane protein 2A |
| 210794_s_at | 3.0748 | 0.244963 | MEG3 | maternally expressed 3 |
| 219985_at | 3.4622 | 0.247231 | HS3ST3A1 | heparan sulfate (glucosamine) 3-O-sulfotransferase 3A1 |
| 205528_s_at | 2.9963 | 0.247231 | RUNX1T1 | NA |
| 215513_at | 2.9132 | 0.252439 | PLAGL1 | pleiomorphic adenoma gene-like 1 |
| 217892_s_at | 2.1450 | 0.252439 | EPLIN | NA |
| 219024_at | 2.1399 | 0.253051 | PLEKHA1 | pleckstrin homology domain containing, family A (phosphoinositide binding specific) |
| | | | | member 1 |
| 219454_at | 7.3884 | 0.255338 | EGFL6 | EGF-like-domain, multiple 6 |
| 201466_s_at | 4.3301 | 0.255948 | JUN | v-jun sarcoma virus 17 oncogene homolog (avian) |
| 203946_s_at | 2.4110 | 0.255948 | ARG2 | arginase, type II |
| 202350_s_at | 2.0377 | 0.255948 | MATN2 | matrilin 2 |
| 207379_at | 6.0076 | 0.259886 | EDIL3 | EGF-like repeats and discoidin I-like domains 3 |
| 205559_s_at | 2.1591 | 0.264244 | PCSK5 | proprotein convertase subtilisin/kexin type 5 |
| 220407_s_at | 4.4432 | 0.26467 | TGFB2 | transforming growth factor, beta 2 |
| 204235_s_at | 2.3966 | 0.26467 | GULP1 | GULP, engulfment adaptor PTB domain containing 1 |
| 201069_at | 2.1143 | 0.26467 | MMP2 | matrix metalloproteinase 2 (gelatinase A, 72 kDa gelatinase, 72 kDa type IV collagenase) |
| 203413_at | 6.3964 | 0.269934 | NELL2 | NEL-like 2 (chicken) |
| 207558_s_at | 3.4530 | 0.279427 | PITX2 | paired-like homeodomain transcription factor 2 |
| 202342_s_at | 2.4639 | 0.279427 | TRIM2 | tripartite motif-containing 2 |
| 209437_s_at | 2.3619 | 0.279427 | SPON1 | spondin 1, extracellular matrix protein |
| 219179_at | 2.9570 | 0.280147 | DACT1 | dapper homolog 1, antagonist of beta-catenin (xenopus) |
| 215501_s_at | 3.1362 | 0.281534 | DUSP10 | dual specificity phosphatase 10 |
| 212445_s_at | 2.5177 | 0.287182 | NEDD4L | neural precursor cell expressed, developmentally down-regulated 4-like |
| 213285_at | 3.1815 | 0.290838 | TMEM30B | transmembrane protein 30B |
| 202289_s_at | 2.4871 | 0.290838 | TACC2 | transforming, acidic coiled-coil containing protein 2 |
| 213779_at | 2.5346 | 0.291019 | EWSR1 | Ewing sarcoma breakpoint region 1 |
| 202746_at | 2.2311 | 0.291019 | ITM2A | integral membrane protein 2A |
| 208228_s_at | 3.3646 | 0.293538 | FGFR2 | fibroblast growth factor receptor 2 (bacteria-expressed kinase, keratinocyte growth factor |
| | | | | receptor, craniofacial dysostosis 1, Crouzon syndrome, Pfeiffer syndrome, Jackson-Weiss |
| | | | | syndrome) |
| 220092_s_at | 2.9176 | 0.303223 | ANTXR1 | anthrax toxin receptor 1 |
| 213222_at | 2.2131 | 0.303223 | PLCB1 | phospholipase C, beta 1 (phosphoinositide-specific) |
| 201464_x_at | 3.2130 | 0.313337 | JUN | v-jun sarcoma virus 17 oncogene homolog (avian) |
| 206132_at | 2.2103 | 0.313337 | MCC | mutated in colorectal cancers |
| 204755_x_at | 2.7655 | 0.319199 | HLF | hepatic leukemia factor |
| 211432_s_at | 2.1877 | 0.320535 | TYRO3 | TYRO3 protein tyrosine kinase |
| 212086_x_at | 2.2897 | 0.326073 | LMNA | lamin A/C |
| 204451_at | 2.1305 | 0.326073 | FZD1 | frizzled homolog 1 (Drosophila) |
| 204326_x_at | 2.0202 | 0.326073 | MT1X | metallothionein 1X |
| 213281_at | 2.7091 | 0.328483 | NA | NA |
| 204237_at | 2.0550 | 0.328483 | GULP1 | GULP, engulfment adaptor PTB domain containing 1 |
| 217525_at | 2.8854 | 0.332607 | OLFML1 | olfactomedin-like 1 |
| 219779_at | 2.0508 | 0.332607 | ZFHX4 | zinc finger homeodomain 4 |
| 219288_at | 2.4542 | 0.336447 | C3orf14 | chromosome 3 open reading frame 14 |
| 205547_s_at | 2.8566 | 0.348362 | TAGLN | transgelin |
| 205809_s_at | 2.4832 | 0.354284 | WASL | Wiskott-Aldrich syndrome-like |
| 209627_s_at | 2.0472 | 0.357677 | OSBPL3 | oxysterol binding protein-like 3 |
| 212845_at | 2.0941 | 0.36137 | SAMD4 | sterile alpha motif domain containing 4 |
| 202965_s_at | 4.9111 | 0.365609 | CAPN6 | calpain 6 |
| 218051_s_at | 3.0570 | 0.365609 | FLJ12442 | NA |
| 209908_s_at | 2.0000 | 0.365609 | TGFB2 | transforming growth factor, beta 2 |
| 203789_s_at | 3.7336 | 0.382939 | SEMA3C | sema domain, immunoglobulin domain (Ig), short basic domain, secreted, (semaphorin) 3C |
| 211401_s_at | 3.2862 | 0.382939 | FGFR2 | fibroblast growth factor receptor 2 (bacteria-expressed kinase, keratinocyte growth factor |
| | | | | receptor, craniofacial dysostosis 1, Crouzon syndrome, Pfeiffer syndrome, Jackson-Weiss |
| | | | | syndrome) |
| 202363_at | 3.4212 | 0.388198 | SPOCK | sparc/osteonectin, cwcv and kazal-like domains proteoglycan (testican) |
| 204679_at | 2.1469 | 0.388198 | KCNK1 | potassium channel, subfamily K, member 1 |
| 213492_at | 7.2139 | 0.390079 | COL2A1 | collagen, type II, alpha 1 (primary osteoarthritis, spondyloepiphyseal dysplasia, congenital) |
| 205116_at | 3.3214 | 0.390079 | LAMA2 | laminin, alpha 2 (merosin, congenital muscular dystrophy) |
| 203411_s_at | 2.3355 | 0.390496 | LMNA | lamin A/C |
| 204324_s_at | 2.1998 | 0.394453 | GOLPH4 | golgi phosphoprotein 4 |
| 212992_at | 2.7425 | 0.397167 | C14orf78 | chromosome 14 open reading frame 78 |
| 202206_at | 2.1425 | 0.397333 | ARL7 | ADP-ribosylation factor-like 7 |
| 219532_at | 2.3428 | 0.399777 | ELOVL4 | elongation of very long chain fatty acids (FEN1/EIo2, SUR4/EIo3, yeast)-like 4 |
| 201430_s_at | 2.1716 | 0.405244 | DPYSL3 | dihydropyrimidinase-like 3 |
| 209747_at | 2.1294 | 0.405244 | TGFB3 | transforming growth factor, beta 3 |
| 217428_s_at | 14.6506 | 0.407137 | COL10A1 | collagen, type X, alpha 1(Schmid metaphyseal chondrodysplasia) |
| 202185_at | 2.0947 | 0.407137 | PLOD3 | procollagen-lysine, 2-oxoglutarate 5-dioxygenase 3 |
| 201141_at | 2.0937 | 0.407137 | GPNMB | glycoprotein (transmembrane) nmb |
| 212865_s_at | 2.1165 | 0.417028 | COL14A1 | collagen, type XIV, alpha 1 (undulin) |
| 212937_s_at | 2.4723 | 0.41747 | COL6A1 | collagen, type VI, alpha 1 |
| 212758_s_at | 2.0645 | 0.4225 | TCF8 | transcription factor 8 (represses interleukin 2 expression) |
| 208850_s_at | 2.5566 | 0.426782 | THY1 | Thy-1 cell surface antigen |
| 45297_at | 2.2019 | 0.428404 | EHD2 | EH-domain containing 2 |
| 209598_at | 3.1634 | 0.438233 | PNMA2 | paraneoplastic antigen MA2 |
| 213869_x_at | 2.2642 | 0.438233 | THY1 | Thy-1 cell surface antigen |
| 207480_s_at | 2.0999 | 0.438233 | MEIS2 | Meis1, myeloid ecotropic viral integration site 1 homolog 2 (mouse) |
| 205348_s_at | 2.1777 | 0.443314 | DNCI1 | dynein, cytoplasmic, intermediate polypeptide 1 |
| 219390_at | 2.0968 | 0.445512 | FKBP14 | FK506 binding protein 14, 22 kDa |
| 205529_s_at | 2.9673 | 0.446359 | RUNX1T1 | NA |
| 209156_s_at | 2.4602 | 0.446421 | COL6A2 | collagen, type VI, alpha 2 |
| 213901_x_at | 2.2475 | 0.449199 | RBM9 | RNA binding motif protein 9 |
| 208711_s_at | 2.1319 | 0.449199 | CCND1 | cyclin D1 (PRAD1: parathyroid adenomatosis 1) |
| 208033_s_at | 2.1224 | 0.449199 | ATBF1 | AT-binding transcription factor 1 |
| 204749_at | 2.0205 | 0.449199 | NAP1L3 | nucleosome assembly protein 1-like 3 |
| 209840_s_at | 2.8579 | 0.457562 | LRRN3 | leucine rich repeat neuronal 3 |
| 203639_s_at | 3.6217 | 0.466813 | FGFR2 | fibroblast growth factor receptor 2 (bacteria-expressed kinase, keratinocyte growth factor |
| | | | | receptor, craniofacial dysostosis 1, Crouzon syndrome, Pfeiffer syndrome, Jackson-Weiss |
| | | | | syndrome) |
| 201149_s_at | 2.2207 | 0.466813 | TIMP3 | tissue inhibitor of metalloproteinase 3 (Sorsby fundus dystrophy, pseudoinflammatory) |
| 221734_at | 2.1478 | 0.466813 | LOC133619 | NA |
| 209542_x_at | 2.1119 | 0.466813 | IGF1 | insulin-like growth factor 1 (somatomedin C) |
| 217771_at | 2.0343 | 0.466813 | GOLPH2 | golgi phosphoprotein 2 |
| 210303_at | 2.0726 | 0.467356 | MAB21L2 | mab-21-like 2 (C. elegans) |
| 213558_at | 2.8365 | 0.46968 | PCLO | piccolo (presynaptic cytomatrix protein) |
| 219729_at | 2.2755 | 0.46968 | PRRX2 | paired related homeobox 2 |
| 217404_s_at | 6.7805 | 0.471203 | COL2A1 | collagen, type II, alpha 1 (primary osteoarthritis, spondyloepiphyseal dysplasia, congenital) |
| 206159_at | 2.5077 | 0.471203 | GDF10 | growth differentiation factor 10 |
| 212089_at | 2.0602 | 0.471203 | LMNA | lamin A/C |
| 209632_at | 2.0075 | 0.471203 | PPP2R3A | protein phosphatase 2 (formerly 2A), regulatory subunit B″, alpha |
| 208851_s_at | 2.2918 | 0.475442 | THY1 | Thy-1 cell surface antigen |
| 202458_at | 2.0273 | 0.475442 | PRSS23 | protease, serine, 23 |
| 221276_s_at | 2.3067 | 0.475999 | SYNC1 | syncoilin, intermediate filament 1 |
| 211000_s_at | 2.0347 | 0.476667 | IL6ST | interleukin 6 signal transducer (gp130, oncostatin M receptor) |
| 209841_s_at | 2.1358 | 0.477181 | LRRN3 | leucine rich repeat neuronal 3 |
| 205560_at | 2.0162 | 0.477181 | PCSK5 | proprotein convertase subtilisin/kexin type 5 |
| 204823_at | 2.0153 | 0.477181 | NAV3 | neuron navigator 3 |
| 205941_s_at | 7.2341 | 0.479823 | COL10A1 | collagen, type X, alpha 1(Schmid metaphyseal chondrodysplasia) |
| 210302_s_at | 3.8896 | 0.479823 | MAB21L2 | mab-21-like 2 (C. elegans) |
| 206326_at | 3.3490 | 0.479823 | GRP | gastrin-releasing peptide |
| 209955_s_at | 3.3479 | 0.479823 | FAP | fibroblast activation protein, alpha |
| 216840_s_at | 3.0463 | 0.479823 | LAMA2 | laminin, alpha 2 (merosin, congenital muscular dystrophy) |
| 204472_at | 2.9523 | 0.479823 | GEM | GTP binding protein overexpressed in skeletal muscle |
| 206071_s_at | 2.9195 | 0.479823 | EPHA3 | EPH receptor A3 |
| 207118_s_at | 2.8202 | 0.479823 | MMP23B | matrix metalloproteinase 23B |
| 211276_at | 2.5109 | 0.479823 | TCEAL2 | transcription elongation factor A (SII)-like 2 |
| 205931_s_at | 2.4242 | 0.479823 | CREB5 | cAMP responsive element binding protein 5 |
| 205206_at | 2.4209 | 0.479823 | KAL1 | Kallmann syndrome 1 sequence |
| 202581_at | 2.4161 | 0.479823 | HSPA1B | heat shock 70 kDa protein 1B |
| 219230_at | 2.3793 | 0.479823 | FLJ10970 | NA |
| 205683_x_at | 2.3736 | 0.479823 | TPSAB1 | tryptase alpha/beta 1 |
| 205475_at | 2.3271 | 0.479823 | SCRG1 | NA |
| 220260_at | 2.2137 | 0.479823 | TBC1D19 | TBC1 domain family, member 19 |
| 207134_x_at | 2.1583 | 0.479823 | TPSB2 | tryptase beta 2 |
| 217023_x_at | 2.1025 | 0.479823 | TPSB2 | tryptase beta 2 |
| 45714_at | 2.0375 | 0.479823 | HCFC1R1 | host cell factor C1 regulator 1 (XPO1 dependant) |
| 216474_x_at | 2.0257 | 0.479823 | TPSAB1 | tryptase alpha/beta 1 |
| 204078_at | 2.0073 | 0.479823 | SC65 | NA |
| 202672_s_at | 2.0980 | 0.514107 | ATF3 | activating transcription factor 3 |
| 206837_at | 2.0581 | 0.532754 | CART1 | CART1lage paired-class homeoprotein 1 |
| 209728_at | 2.2906 | 0.54295 | HLA-DRB1 | major histocompatibility complex, class II, DR beta 1 |

TABLE 3
Up-regulated in Fused sutures and bone cell differentiation ie. Promotes osteoblast differentiation and mineralization:

| ID | Fold Change | P. Value | Gene | Name |
|---|---|---|---|---|
| 220999_s_at | −2.68296 | 0.031356 | CYFIP2 | cytoplasmic FMR1 interacting protein 2 |
| 204567_s_at | −2.24569 | 0.096126 | ABCG1 | ATP-binding cassette, sub-family G (WHITE), member 1 |
| 203980_at | −3.52254 | 0.10987 | FABP4 | fatty acid binding protein 4, adipocyte |
| 204646_at | −2.61853 | 0.120427 | DPYD | dihydropyrimidine dehydrogenase |
| 210135_s_at | −3.438452848 | 0.479822977 | SHOX2 | short stature homeobox |
| 213566_at | −2.56681 | 0.145863 | RNASE6 | ribonuclease, RNase A family, k6 |
| 218829_s_at | −2.12126 | 0.16387 | CHD7 | chromodomain helicase DNA binding protein 7 |
| 215071_s_at | −3.14812 | 0.164269 | HIST1H1E | histone 1, H1e |
| 211366_x_at | −2.72612 | 0.165282 | CASP1 | caspase 1, apoptosis-related cysteine protease (interleukin 1, beta, convertase) |
| 204236_at | −2.18989 | 0.165282 | FLI1 | Friend leukemia virus integration 1 |
| 219870_at | −2.29856 | 0.178705 | ATF7IP2 | activating transcription factor 7 interacting protein 2 |
| 201998_at | −2.37348 | 0.194086 | ST6GAL1 | ST6 beta-galactosamide alpha-2,6-sialyltranferase 1 |
| 203414_at | −2.02131 | 0.204541 | MMD | monocyte to macrophage differentiation-associated |
| 213142_x_at | −3.19415 | 0.219174 | LOC54103 | NA |
| 221840_at | −2.99286 | 0.225122 | PTPRE | protein tyrosine phosphatase, receptor type, E |
| 206060_s_at | −2.75084 | 0.227497 | PTPN22 | protein tyrosine phosphatase, non-receptor type 22 (lymphoid) |
| 202687_s_at | −2.26373 | 0.244105 | TNFSF10 | tumor necrosis factor (ligand) superfamily, member 10 |
| 219681_s_at | −2.15094 | 0.252439 | RAB11FIP1 | RAB11 family interacting protein 1 (class I) |
| 203359_s_at | −2.21755 | 0.253051 | MYCBP | c-myc binding protein |
| 211367_s_at | −2.1831 | 0.253051 | CASP1 | caspase 1, apoptosis-related cysteine protease (interleukin 1, beta, convertase) |
| 203185_at | −3.30012 | 0.253663 | RASSF2 | Ras association (RalGDS/AF-6) domain family 2 |
| 216899_s_at | −3.09217 | 0.254277 | SCAP2 | src family associated phosphoprotein 2 |
| 215933_s_at | −3.752 | 0.26355 | HHEX | hematopoietically expressed homeobox |
| 205440_s_at | −2.19509 | 0.269256 | NA | NA |
| 215049_x_at | −2.18309 | 0.279409 | CD163 | CD163 antigen |
| 209574_s_at | −3.21351 | 0.279427 | C18orf1 | chromosome 18 open reading frame 1 |
| 210951_x_at | −2.44302 | 0.279427 | RAB27A | RAB27A, member RAS oncogene family |
| 222150_s_at | −3.05948 | 0.283071 | LOC54103 | NA |
| 205798_at | −2.68125 | 0.287182 | IL7R | interleukin 7 receptor |
| 213094_at | −2.32086 | 0.287182 | GPR126 | G protein-coupled receptor 126 |
| 206637_at | −3.72737 | 0.288375 | P2RY14 | purinergic receptor P2Y, G-protein coupled, 14 |
| 218870_at | −3.99044 | 0.291019 | ARHGAP15 | Rho GTPase activating protein 15 |
| 204689_at | −2.62511 | 0.291019 | HHEX | hematopoietically expressed homeobox |
| 209970_x_at | −2.39581 | 0.292614 | CASP1 | caspase 1, apoptosis-related cysteine protease (interleukin 1, beta, convertase) |
| 204232_at | −2.3949 | 0.313337 | FCER1G | NA |
| 202388_at | −2.19904 | 0.313337 | RGS2 | regulator of G-protein signaling 2, 24 kDa |
| 203932_at | −2.01247 | 0.313337 | HLA-DMB | major histocompatibility complex, class II, DM beta |
| 202430_s_at | −2.07266 | 0.326073 | PLSCR1 | phospholipid scramblase 1 |
| 202446_s_at | −2.06764 | 0.326073 | PLSCR1 | phospholipid scramblase 1 |
| 213293_s_at | −2.5709 | 0.328483 | TRIM22 | tripartite motif-containing 22 |
| 220147_s_at | −2.21791 | 0.328483 | FAM60A | family with sequence similarity 60, member A |
| 209933_s_at | −2.47518 | 0.332607 | CD300A | CD300A antigen |
| 205733_at | −2.72477 | 0.333245 | BLM | Bloom syndrome |
| 219033_at | −2.37233 | 0.333245 | PARP8 | poly (ADP-ribose) polymerase family, member 8 |
| 201720_s_at | −2.91801 | 0.337757 | LAPTM5 | lysosomal associated multispanning membrane protein 5 |
| 213375_s_at | −2.50802 | 0.341916 | CG018 | NA |
| 206235_at | −2.49087 | 0.342699 | LIG4 | ligase IV, DNA, ATP-dependent |
| 207419_s_at | −4.34051 | 0.344254 | RAC2 | ras-related C3 botulinum toxin substrate 2 (rho family, small GTP binding protein |
| | | | | Rac2) |
| 219622_at | −2.08592 | 0.344946 | RAB20 | RAB20, member RAS oncogene family |
| 214791_at | −2.61191 | 0.346085 | LOC93349 | NA |
| 203986_at | −2.12187 | 0.349546 | GENX- | NA |
| | | | 3414 | |
| 221586_s_at | −2.49836 | 0.354284 | E2F5 | E2F transcription factor 5, p130-binding |
| 207761_s_at | −2.41639 | 0.354284 | DKFZP586A0522 | NA |
| 215785_s_at | −2.26065 | 0.358015 | CYFIP2 | cytoplasmic FMR1 interacting protein 2 |
| 207275_s_at | −2.41347 | 0.36137 | ACSL1 | acyl-CoA synthetase long-chain family member 1 |
| 204972_at | −2.23108 | 0.36137 | OAS2 | 2′-5′-oligoadenylate synthetase 2, 69/71 kDa |
| 203474_at | −4.19404 | 0.362553 | IQGAP2 | IQ motif containing GTPase activating protein 2 |
| 220132_s_at | −2.89418 | 0.362922 | CLEC2D | C-type lectin superfamily 2, member D |
| 217478_s_at | −2.84497 | 0.362922 | HLA-DMA | major histocompatibility complex, class II, DM alpha |
| 211368_s_at | −2.55414 | 0.362922 | CASP1 | caspase 1, apoptosis-related cysteine protease (interleukin 1, beta, convertase) |
| 205739_x_at | −2.3848 | 0.362922 | ZNF588 | zinc finger protein 588 |
| 210786_s_at | −2.68444 | 0.365617 | FLI1 | Friend leukemia virus integration 1 |
| 203416_at | −3.1853 | 0.36985 | CD53 | CD53 antigen |
| 203908_at | −2.18625 | 0.370586 | SLC4A4 | solute carrier family 4, sodium bicarbonate cotransporter, member 4 |
| 201839_s_at | −2.77216 | 0.381879 | TACSTD1 | tumor-associated calcium signal transducer 1 |
| 209201_x_at | −6.68823 | 0.382939 | CXCR4 | chemokine (C—X—C motif) receptor 4 |
| 205442_at | −3.06967 | 0.382939 | MFAP3L | microfibrillar-associated protein 3-like |
| 204924_at | −2.65084 | 0.388198 | TLR2 | toll-like receptor 2 |
| 205270_s_at | −2.42948 | 0.388198 | LCP2 | lymphocyte cytosolic protein 2 (SH2 domain containing leukocyte |
| | | | | protein of 76 kDa) |
| 205269_at | −3.31656 | 0.390079 | LCP2 | lymphocyte cytosolic protein 2 (SH2 domain containing leukocyte |
| | | | | protein of 76 kDa) |
| 204674_at | −6.54394 | 0.394453 | LRMP | lymphoid-restricted membrane protein |
| 207008_at | −3.08272 | 0.394453 | IL8RB | interleukin 8 receptor, beta |
| 206011_at | −2.58899 | 0.394453 | CASP1 | caspase 1, apoptosis-related cysteine protease (interleukin 1, beta, convertase) |
| 209515_s_at | −2.43075 | 0.397333 | RAB27A | RAB27A, member RAS oncogene family |
| 212738_at | −2.24938 | 0.397333 | ARHGAP19 | Rho GTPase activating protein 19 |
| 211919_s_at | −6.90408 | 0.399777 | CXCR4 | chemokine (C—X—C motif) receptor 4 |
| 210785_s_at | −3.24444 | 0.399777 | C1orf38 | chromosome 1 open reading frame 38 |
| 207571_x_at | −2.50627 | 0.399777 | C1orf38 | chromosome 1 open reading frame 38 |
| 222294_s_at | −2.05856 | 0.399777 | RAB27A | RAB27A, member RAS oncogene family |
| 219694_at | −2.0132 | 0.399777 | FLJ11127 | NA |
| 205668_at | −3.14785 | 0.407137 | LY75 | lymphocyte antigen 75 |
| 210982_s_at | −2.62089 | 0.407137 | HLA-DRA | major histocompatibility complex, class II, DR alpha |
| 210449_x_at | −2.31683 | 0.407137 | MAPK14 | mitogen-activated protein kinase 14 |
| 207238_s_at | −4.57987 | 0.40934 | PTPRC | protein tyrosine phosphatase, receptor type, C |
| 203761_at | −3.72358 | 0.410093 | SLA | Src-like-adaptor |
| 204160_s_at | −2.07296 | 0.413198 | ENPP4 | ectonucleotide pyrophosphatase/phosphodiesterase 4 (putative function) |
| 204613_at | −2.47698 | 0.413332 | PLCG2 | phospholipase C, gamma 2 (phosphatidylinositol-specific) |
| 218640_s_at | −2.11268 | 0.417028 | PLEKHF2 | pleckstrin homology domain containing, family F (with FYVE domain) member 2 |
| 209238_at | −2.49432 | 0.42386 | STX3A | syntaxin 3A |
| 213618_at | −3.27055 | 0.428404 | CENTD1 | centaurin, delta 1 |
| 220235_s_at | −2.30834 | 0.428404 | RIF1 | NA |
| 211561_x_at | −2.36204 | 0.434122 | MAPK14 | mitogen-activated protein kinase 14 |
| 212588_at | −3.57617 | 0.438233 | PTPRC | protein tyrosine phosphatase, receptor type, C |
| 208894_at | −2.70778 | 0.438233 | HLA-DRA | major histocompatibility complex, class II, DR alpha |
| 202112_at | −2.05058 | 0.438233 | VWF | von Willebrand factor |
| 204407_at | −2.12989 | 0.446108 | TTF2 | transcription termination factor, RNA polymerase II |
| 214475_x_at | −2.07275 | 0.446108 | CAPN3 | calpain 3, (p94) |
| 216920_s_at | −2.7952 | 0.447995 | TARP | NA |
| 203680_at | −3.86136 | 0.449199 | PRKAR2B | protein kinase, cAMP-dependent, regulatory, type II, beta |
| 211742_s_at | −3.16561 | 0.449199 | EVI2B | ecotropic viral integration site 2B |
| 203608_at | −2.88109 | 0.449199 | GPLD1 | glycosylphosphatidylinositol specific phospholipase D1 |
| 212998_x_at | −2.86342 | 0.449199 | HLA-DQB1 | major histocompatibility complex, class II, DQ beta 1 |
| 210004_at | −2.78445 | 0.449199 | OLR1 | oxidised low density lipoprotein (lectin-like) receptor 1 |
| 219079_at | −2.69903 | 0.449199 | NCB5OR | NADPH cytochrome B5 oxidoreductase |
| 221210_s_at | −2.57789 | 0.449199 | NPL | N-acetylneuraminate pyruvate lyase (dihydrodipicolinate synthase) |
| 205316_at | −2.30632 | 0.449199 | SLC15A2 | solute carrier family 15 (H+/peptide transporter), member 2 |
| 201688_s_at | −2.09635 | 0.449199 | TPD52 | tumor protein D52 |
| 209514_s_at | −2.0618 | 0.449199 | RAB27A | RAB27A, member RAS oncogene family |
| 208885_at | −3.16251 | 0.451893 | LCP1 | lymphocyte cytosolic protein 1 (L-plastin) |
| 209734_at | −3.08017 | 0.451893 | HEM1 | hematopoietic protein 1 |
| 209040_s_at | −2.55987 | 0.451893 | PSMB8 | proteasome (prosome, macropain) subunit, beta type, 8 (large multifunctional |
| | | | | protease 7) |
| 204951_at | −3.37825 | 0.453119 | RHOH | ras homolog gene family, member H |
| 204006_s_at | −4.19241 | 0.455174 | FCGR3B | Fc fragment of IgG, low affinity IIIb, receptor (CD16b) |
| 206478_at | −3.94183 | 0.455174 | KIAA0125 | KIAA0125 |
| 203372_s_at | −2.55891 | 0.455174 | SOCS2 | suppressor of cytokine signaling 2 |
| 204510_at | −2.52034 | 0.455174 | CDC7 | CDC7 cell division cycle 7 (S. cerevisiae) |
| 218949_s_at | −2.3747 | 0.455174 | QRSL1 | glutaminyl-tRNA synthase (glutamine-hydrolyzing)-like 1 |
| 204362_at | −2.32784 | 0.459056 | SCAP2 | src family associated phosphoprotein 2 |
| 219191_s_at | −3.24193 | 0.463597 | BIN2 | bridging integrator 2 |
| 204220_at | −2.9703 | 0.464815 | GMFG | glia maturation factor, gamma |
| 221581_s_at | −2.56843 | 0.464815 | WBSCR5 | Williams-Beuren syndrome chromosome region 5 |
| 202990_at | −2.54767 | 0.464815 | PYGL | NA |
| 221523_s_at | −2.31704 | 0.466406 | RRAGD | Ras-related GTP binding D |
| 217028_at | −3.93007 | 0.466813 | CXCR4 | chemokine (C—X—C motif) receptor 4 |
| 205681_at | −3.41606 | 0.466813 | BCL2A1 | BCL2-related protein A1 |
| 210164_at | −3.27432 | 0.466813 | GZMB | granzyme B (granzyme 2, cytotoxic T-lymphocyte-associated serine esterase 1) |
| 204279_at | −2.93971 | 0.466813 | PSMB9 | proteasome (prosome, macropain) subunit, beta type, 9 (large multifunctional |
| | | | | protease 2) |
| 204240_s_at | −2.33745 | 0.466813 | SMC2L1 | SMC2 structural maintenance of chromosomes 2-like 1 (yeast) |
| 206206_at | −2.15886 | 0.466813 | LY64 | lymphocyte antigen 64 homolog, radioprotective 105 kDa (mouse) |
| 210153_s_at | −2.14532 | 0.466813 | ME2 | malic enzyme 2, NAD(+)-dependent, mitochondrial |
| 220651_s_at | −2.03924 | 0.466813 | MCM10 | MCM10 minichromosome maintenance deficient 10 (S. cerevisiae) |
| 207992_s_at | −4.58777 | 0.468467 | AMPD3 | adenosine monophosphate deaminase (isoform E) |
| 207072_at | −3.66256 | 0.46968 | IL18RAP | interleukin 18 receptor accessory protein |
| 220005_at | −3.5106 | 0.46968 | P2RY13 | purinergic receptor P2Y, G-protein coupled, 13 |
| 216491_x_at | −3.49975 | 0.46968 | IGHG1 | immunoglobulin heavy constant gamma 1 (G1m marker) |
| 203665_at | −3.37013 | 0.46968 | HMOX1 | heme oxygenase (decycling) 1 |
| 208699_x_at | −3.17022 | 0.46968 | TKT | transketolase (Wernicke-Korsakoff syndrome) |
| 222071_s_at | −2.96213 | 0.46968 | SLCO4C1 | solute carrier organic anion transporter family, member 4C1 |
| 217148_x_at | −2.92067 | 0.46968 | IGLC2 | immunoglobulin lambda constant 2 (Kern-Oz-marker) |
| 209606_at | −2.9176 | 0.46968 | PSCDBP | pleckstrin homology, Sec7 and coiled-coil domains, binding protein |
| 216984_x_at | −2.20641 | 0.46968 | IGLC2 | immunoglobulin lambda constant 2 (Kern-Oz-marker) |
| 207957_s_at | −5.59006 | 0.471203 | PRKCB1 | protein kinase C, beta 1 |
| 35974_at | −5.18914 | 0.471203 | LRMP | lymphoid-restricted membrane protein |
| 202191_s_at | −3.30363 | 0.471203 | GAS7 | growth arrest-specific 7 |
| 205041_s_at | −3.19313 | 0.471203 | ORM1 | orosomucoid 1 |
| 206978_at | −2.94568 | 0.471203 | CCR2 | chemokine (C-C motif) receptor 2 |
| 210262_at | −2.53812 | 0.471203 | CRISP2 | cysteine-rich secretory protein 2 |
| 219863_at | −2.48413 | 0.471203 | HERC5 | hect domain and RLD 5 |
| 213599_at | −2.40784 | 0.471203 | OIP5 | Opa interacting protein 5 |
| 204361_s_at | −2.39825 | 0.471203 | SCAP2 | src family associated phosphoprotein 2 |
| 205184_at | −2.37012 | 0.471203 | GNG4 | guanine nucleotide binding protein (G protein), gamma 4 |
| 219510_at | −2.30184 | 0.471203 | POLQ | polymerase (DNA directed), theta |
| 211991_s_at | −2.14323 | 0.471203 | HLA-DPA1 | major histocompatibility complex, class II, DP alpha 1 |
| 219607_s_at | −2.06396 | 0.471203 | MS4A4A | membrane-spanning 4-domains, subfamily A, member 4 |
| 201678_s_at | −2.05011 | 0.471203 | DC12 | NA |
| 218454_at | −2.96731 | 0.475442 | FLJ22662 | NA |
| 211339_s_at | −2.08332 | 0.475442 | ITK | IL2-inducible T-cell kinase |
| 204982_at | −2.69627 | 0.475809 | GIT2 | G protein-coupled receptor kinase interactor 2 |
| 201690_s_at | −3.58442 | 0.475999 | TPD52 | tumor protein D52 |
| 209269_s_at | −3.24841 | 0.475999 | SYK | spleen tyrosine kinase |
| 204224_s_at | −2.79677 | 0.475999 | GCH1 | GTP cyclohydrolase 1 (dopa-responsive dystonia) |
| 205040_at | −2.78434 | 0.475999 | ORM1 | orosomucoid 1 |
| 203820_s_at | −2.50853 | 0.475999 | IMP-3 | NA |
| 209795_at | −2.88006 | 0.476052 | CD69 | CD69 antigen (p60, early T-cell activation antigen) |
| 203434_s_at | −3.23216 | 0.476293 | MME | membrane metallo-endopeptidase (neutral endopeptidase, enkephalinase, CALLA, |
| | | | | CD10) |
| 205039_s_at | −2.95421 | 0.476349 | ZNFN1A1 | zinc finger protein, subfamily 1A, 1 (lkaros) |
| 220603_s_at | −2.40341 | 0.476368 | MCTP2 | NA |
| 210356_x_at | −3.96843 | 0.476667 | MS4A1 | membrane-spanning 4-domains, subfamily A, member 1 |
| 213603_s_at | −2.88903 | 0.476667 | RAC2 | ras-related C3 botulinum toxin substrate 2 (rho family, small GTP binding protein |
| | | | | Rac2) |
| 209498_at | −5.10907 | 0.477181 | CEACAM1 | carcinoembryonic antigen-related cell adhesion molecule 1 (biliary glycoprotein) |
| 220416_at | −4.64032 | 0.477181 | ATP8B4 | ATPase, Class I, type 8B, member 4 |
| 204698_at | −3.95179 | 0.477181 | ISG20 | interferon stimulated gene 20 kDa |
| 206283_s_at | −3.71503 | 0.477181 | TAL1 | T-cell acute lymphocytic leukemia 1 |
| 205692_s_at | −3.6578 | 0.477181 | CD38 | CD38 antigen (p45) |
| 215117_at | −3.60688 | 0.477181 | RAG2 | recombination activating gene 2 |
| 202157_s_at | −3.21265 | 0.477181 | CUGBP2 | CUG triplet repeat, RNA binding protein 2 |
| 207655_s_at | −3.02601 | 0.477181 | BLNK | B-cell linker |
| 203126_at | −2.76382 | 0.477181 | IMPA2 | inositol(myo)-1(or 4)-monophosphatase 2 |
| 205984_at | −2.65601 | 0.477181 | CRHBP | corticotropin releasing hormone binding protein |
| 204887_s_at | −2.61199 | 0.477181 | PLK4 | polo-like kinase 4 (Drosophila) |
| 210154_at | −2.55573 | 0.477181 | ME2 | malic enzyme 2, NAD(+)-dependent, mitochondrial |
| 201288_at | −2.54872 | 0.477181 | ARHGDIB | Rho GDP dissociation inhibitor (GDI) beta |
| 206641_at | −2.38011 | 0.477181 | TNFRSF17 | tumor necrosis factor receptor superfamily, member 17 |
| 212949_at | −2.37493 | 0.477181 | BRRN1 | barren homolog (Drosophila) |
| 201721_s_at | −2.36367 | 0.477181 | LAPTM5 | lysosomal associated multispanning membrane protein 5 |
| 209619_at | −2.18813 | 0.477181 | CD74 | CD74 antigen (invariant polypeptide of major histocompatibility complex, class II |
| | | | | antigen-associated) |
| 209901_x_at | −2.08385 | 0.477181 | AIF1 | allograft inflammatory factor 1 |
| 202519_at | −2.02251 | 0.479133 | MONDOA | NA |
| 203418_at | −2.68756 | 0.479222 | CCNA2 | cyclin A2 |
| 206207_at | −9.96112 | 0.479823 | CLC | Charcot-Leyden crystal protein |
| 205863_at | −8.85883 | 0.479823 | S100A12 | S100 calcium binding protein A12 (calgranulin C) |
| 206676_at | −8.5483 | 0.479823 | CEACAM8 | carcinoembryonic antigen-related cell adhesion molecule 8 |
| 219014_at | −7.1933 | 0.479823 | PLAC8 | placenta-specific 8 |
| 209369_at | −7.17324 | 0.479823 | ANXA3 | annexin A3 |
| 212560_at | −7.06618 | 0.479823 | SORL1 | sortilin-related receptor, L(DLR class) A repeats-containing |
| 202018_s_at | −6.89731 | 0.479823 | LTF | lactotransferrin |
| 205267_at | −6.86529 | 0.479823 | POU2AF1 | POU domain, class 2, associating factor 1 |
| 212531_at | −6.75808 | 0.479823 | LCN2 | lipocalin 2 (oncogene 24p3) |
| 212768_s_at | −6.69072 | 0.479823 | OLFM4 | olfactomedin 4 |
| 207802_at | −6.62206 | 0.479823 | CRISP3 | cysteine-rich secretory protein 3 |
| 203949_at | −6.49571 | 0.479823 | MPO | myeloperoxidase |
| 209995_s_at | −6.31374 | 0.479823 | TCL1A | T-cell leukemia/lymphoma 1A |
| 203948_s_at | −6.01372 | 0.479823 | MPO | myeloperoxidase |
| 210487_at | −5.94988 | 0.479823 | DNTT | deoxynucleotidyltransferase, terminal |
| 207341_at | −5.92155 | 0.479823 | PRTN3 | proteinase 3 (serine proteinase, neutrophil, Wegener granulomatosis autoantigen) |
| 203535_at | −5.88438 | 0.479823 | S100A9 | S100 calcium binding protein A9 (calgranulin B) |
| 210254_at | −5.87243 | 0.479823 | MS4A3 | membrane-spanning 4-domains, subfamily A, member 3 (hematopoietic cell- |
| | | | | specific) |
| 206851_at | −5.8666 | 0.479823 | RNASE3 | ribonuclease, RNase A family, 3 (eosinophil cationic protein) |
| 207329_at | −5.81357 | 0.479823 | MMP8 | matrix metalloproteinase 8 (neutrophil collagenase) |
| 204959_at | −5.80599 | 0.479823 | MNDA | myeloid cell nuclear differentiation antigen |
| 204563_at | −5.63686 | 0.479823 | SELL | selectin L (lymphocyte adhesion molecule 1) |
| 204446_s_at | −5.54504 | 0.479823 | ALOX5 | arachidonate 5-lipoxygenase |
| 208470_s_at | −5.52664 | 0.479823 | HP | haptoglobin |
| 204466_s_at | −5.51565 | 0.479823 | SNCA | synuclein, alpha (non A4 component of amyloid precursor) |
| 210244_at | −5.4871 | 0.479823 | CAMP | cathelicidin antimicrobial peptide |
| 205237_at | −5.4459 | 0.479823 | FCN1 | ficolin (collagen/fibrinogen domain containing) 1 |
| 206177_s_at | −5.34982 | 0.479823 | ARG1 | arginase, liver |
| 203757_s_at | −5.31342 | 0.479823 | CEACAM6 | carcinoembryonic antigen-related cell adhesion molecule 6 (non-specific cross |
| | | | | reacting antigen) |
| 203765_at | −5.30031 | 0.479823 | GCA | grancalcin, EF-hand calcium binding protein |
| 204798_at | −5.28694 | 0.479823 | MYB | v-myb myeloblastosis viral oncogene homolog (avian) |
| 206111_at | −5.23035 | 0.479823 | RNASE2 | ribonuclease, RNase A family, 2 (liver, eosinophil-derived neurotoxin) |
| 209374_s_at | −5.1606 | 0.479823 | IGHM | immunoglobulin heavy constant mu |
| 204562_at | −5.15854 | 0.479823 | IRF4 | interferon regulatory factor 4 |
| 206591_at | −5.14455 | 0.479823 | RAG1 | recombination activating gene 1 |
| 204007_at | −5.11461 | 0.479823 | FCGR3B | Fc fragment of IgG, low affinity IIIb, receptor (CD16b) |
| 205513_at | −5.08959 | 0.479823 | TCN1 | transcobalamin I (vitamin B12 binding protein, R binder family) |
| 39318_at | −5.08681 | 0.479823 | TCL1A | T-cell leukemia/lymphoma 1A |
| 209083_at | −5.08208 | 0.479823 | CORO1A | coronin, actin binding protein, 1A |
| 206937_at | −5.06549 | 0.479823 | SPTA1 | spectrin, alpha, erythrocytic 1 (elliptocytosis 2) |
| 211657_at | −5.03854 | 0.479823 | CEACAM6 | carcinoembryonic antigen-related cell adhesion molecule 6 (non-specific cross |
| | | | | reacting antigen) |
| 220001_at | −4.91886 | 0.479823 | PADI4 | peptidyl arginine deiminase, type IV |
| 204971_at | −4.90223 | 0.479823 | CSTA | cystatin A (stefin A) |
| 206390_x_at | −4.81093 | 0.479823 | PF4 | platelet factor 4 (chemokine (C—X—C motif) ligand 4) |
| 211820_x_at | −4.76242 | 0.479823 | GYPA | glycophorin A (includes MN blood group) |
| 204192_at | −4.74305 | 0.479823 | CD37 | CD37 antigen |
| 204351_at | −4.73174 | 0.479823 | S100P | S100 calcium binding protein P |
| 209949_at | −4.65214 | 0.479823 | NCF2 | neutrophil cytosolic factor 2 (65 kDa, chronic granulomatous disease, autosomal 2) |
| 205837_s_at | −4.57742 | 0.479823 | GYPA | glycophorin A (includes MN blood group) |
| 201858_s_at | −4.5118 | 0.479823 | PRG1 | proteoglycan 1, secretory granule |
| 211560_s_at | −4.51132 | 0.479823 | ALAS2 | aminolevulinate, delta-, synthase 2 (sideroblastic/hypochromic anemia) |
| 209480_at | −4.43729 | 0.479823 | HLA-DQB1 | major histocompatibility complex, class II, DQ beta 1 |
| 206515_at | −4.39967 | 0.479823 | CYP4F3 | cytochrome P450, family 4, subfamily F, polypeptide 3 |
| 204174_at | −4.36671 | 0.479823 | ALOX5AP | arachidonate 5-lipoxygenase-activating protein |
| 206522_at | −4.35833 | 0.479823 | MGAM | maltase-glucoamylase (alpha-glucosidase) |
| 206660_at | −4.35624 | 0.479823 | IGLL1 | immunoglobulin lambda-like polypeptide 1 |
| 212827_at | −4.34229 | 0.479823 | IGHM | immunoglobulin heavy constant mu |
| 219471_at | −4.32955 | 0.479823 | C13orf18 | chromosome 13 open reading frame 18 |
| 221349_at | −4.28526 | 0.479823 | VPREB1 | pre-B lymphocyte gene 1 |
| 204419_x_at | −4.20829 | 0.479823 | HBG2 | hemoglobin, gamma G |
| 209395_at | −4.20745 | 0.479823 | CHI3L1 | chitinase 3-like 1 (CART1lage glycoprotein-39) |

TABLE 4
Preferred biomarkers identified

| | Suture | Fusion stage | |
|---|---|---|---|
| GENE | expression | expression | Localization |
| GPC3 | L = C > S | U > Fg = F | membrane |
| RBP4 | L = C > S | U > Fg > F | cytoplasm |
| C1QTNF3 | L = C > S | U > Fg = F | cytoplasm |
| ANXA3 | L = C = S | F > Fg > U | cytoplasm |
| WIF1 | C = L < S | F > Fg > U | EC |
| CASP1 | L = C = S | F > Fg > U | intracellular |

C = coronal suture; L, lambdoid suture; S, sagittal suture; F, fused; Fg, fusing; U, unfused.

One set of preferred biomarkers, GPC3, RBP4 and C1QTNF3, is more highly expressed in unfused sutures whereas another set of preferred biomarkers, ANXA3, WIF1 and CASP1, is more highly expressed in fused and fusing sutures.

TABLE 5
Profile of several preferred biomarkers in human bone cancer cell lines

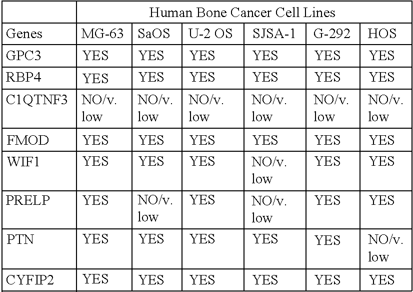

The relative expression of several preferred biomarker genes (highlighted). GPC3, RBPP4, C1QTNF3 and WIF1 (see Table 4) and four other biomarkers. FMOD, PRELP, PTN and CYFIP2 (see Tables 2 and 3) in human bone cancer cell lines were compared to cyclophilin A by real-time RT-PCR. “YES” indicates expression. “NO/v. low” indicates expression was not detectable. The human osteosarcoma cell lines are SaOS (American Type Culture Collection [ATCC] HTB-85), SJSA-1 (ATCC CRL-2098), U-2 OS (HTB-96), MG-63 (ATCC CR1-1427), HOS (ATCC CRL-1543) and G-292 (ATCC CRL-1423).

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Adams, In Vivo 12:11-21, 1998
Akimaru et al, Cytokines Mol Ther 1:197-210, 1995
Alving et al, Immunol Rev 145:5-31, 1995
Amersham International plc sequencing handbook
Angerer, Meth. Enzymol 152:649, 1987
Angew, Chem. Intl. Ed. English 30:423, 1991
Anglian Biotechnology Ltd Handbook
Antibody Conjugate, U.S. Pat. No. 5,106,951
Ausubel et al, Current Protocols in Molecular Biology, F, Greene Publishing and Wiley-Interscience, New York, 1987
Baines et al, Methods in Molecular Biology 10:79-104, 1992
Balcerzak et al, FEBS Lett. 580:3065-9, 2006.
Bandyopadhyay and Temin, Mol Cell Biol 4:749-754, 1984
Barringer et al, Gene 89:117, 1990
Beaucage et al, Tetrahedron 49(10):1925, 1993
Berglund et al, Biotechnology 11:916-920, 1993
Berkner et al, BioTechniques 6:616-629, 1988
Berkner, Curr Top Microbiol Immunol 158:39-66, 1992
Bird et al, Science 242:424-426, 1988
Breakefield and Geller, Mol Neurobiol 1:339-371, 1987
Brinkmann et al, Proc. Natl, Acad. Sci. USA, 90:547-551, 1993
Briu et al, J. Am. Chem. Soc. 111:2321, 1989
Buchschacher and Panganiban, J Virol 66:2731-2739, 1982
Chapters 2 and 3, ASC Symposium Series 580, Carbohydrate Modifications in Antisense Research, Ed. Y S Sanghui and P Dan Cook
Chapters 6 and 7, ASC Symposium Series 580, Carbohydrate Modifications in Antisense Research, Ed. Y S Sanghui and P Dan Cook
Chee, Science 274:610, 1995
Chiswell and McCafferty, J. Tibtech 10:80-84, 1992
Cohen Am J Med Genet. 47 (5):581-616, 1993
Coligan et al, Current Protocols in Immunology 1, John Wiley & Sons 1991
Courtneay-Luck, Monoclonal Antibodies: Production, Engineering and Clinical Application, Ritter et al (eds), Cambridge University Press:166, 1995
Cowan et al, Tissue Eng. 11(3-4):645-658, 2005
De Cat et al, J. Cell Biol. 163(3):625-635, 2003
Denpcy et al, Proc. Natl. Acad. Sci. USA, 92:6097, 1995
Dixon et al, Fundamentals of Craniofacial Growth, New York, CRC Press 1997
Douillard and Hoffman, Basic Facts about Hybridomas, in Compendium of Immunology Vol. II, ed. by Schwartz, 1981
Ducy et al, Cell 89(5):747-754, 1997
Duetscher, Methods in Enzymology Vol. 182: Guide to Protein Purification, Academic Press, Inc. NY, 1990
Eckstein, Oligonucleotides and Analogues: A Practical Approach, Oxford University Press
Edelman et al, Enzymology 1:422, 1967
Egleton and Davis, Peptides 18:1431-1439, 1997
Enlow, Facial Growth, Philadelphia, WB Saunders Co., 1990
Fink et al, Ann Rev Neurosci 19:265-287, 1996
Fink et al, Hum Gene Ther 3:1-19, 1992
Fix, Pharm Res 13:1760-1764, 1996
Fluorogenic Materials and Labeling Techniques U.S. Pat. No. 4,018,884
Freese et al, Biochem Pharmaco. 40:2189-2199, 1990
Furtwangler et al, Acta Anat 124(1-2):74-80, 1985
Gardner et al, Int. J. Epidemiol 27(1):64-67, 1998
Goldenberg et al, International Patent Publication No. WO 91/11465, 1991
Goldenberg U.S. Pat. No. 4,331,647
Gorziglia and Kapikian, J Virol 66:4407-4412, 1992
Green et al, Immunochemical Protocols (Manson ed):1-5, 1992
Green et al, Nature Genet. 7:1994
Guatelli et al, Proc. Nat. Acad. Sci. USA 87:1874, 1990
Helseth et al, J Virol 64:2416-2420, 1990
Hiltunen, Acta Oncol. 32:931-939, 1993
Hodgson, BioTechnology 9:19-21, 1991
Huston et al, Proc. Nat. Acad. Sci. USA, 85:5879-5883, 1988
Inman, Methods In Enzymology 34:30, 1974
Innis et al, PCR Protocols, A Guide to Methods and Application. Academic Press, Inc. San Diego, 1990
International Patent Publication Number WO 96/11698
Jackson, Nature Biotechnology 14:1685, 1996
Jeffs et al, J. Biomolecular NMR 34:17, 1994
Jenkins et al, Chem. Soc. Rev.:169-176, 1995
Johnson et al, J Virol 66:2952-2965, 1992
Jones et al, Nature 321:522-525, 1986
Kohler and Milstein, Nature 256: 495-499, 1975
Kohler and Milstein, European Journal of Immunology 6: 511-519, 1976
Kramer et al, Nucleic Acids Res. 12:9441, 1984
Kunkel, Proc. Natl. Acad. Sci. USA 82:488-492, 1985
Kunkel et al, Methods in Enzymol. 154:367-382, 1987
Kwoh et al, Proc. Natl. Acad. Sci. USA 86:1173, 1989
Landegren et al, Science 241:1077, 1988
Langer, Science 249:1527-1533, 1990
Larrick et al, Methods: A Companion to Methods in Enzymology 2:106, 1991
Lausen et al, Biol. Chem. 274(45):32234-32240, 1999
Letsinger, J. Org. Chem. 35:3800, 1970
Letsinger et al, Nucl. Acids Res. 14:3487, 1986
Letsinger et al, J. Am. Chem. Soc. 110:4470, 1988
Letsinger et al, Nucleoside & Nucleotide 13:1597, 1994
Liu et al, Proc. Natl. Acad. Sci. USA 84:3439-3443, 1987
Lonberg et al, Nature 368:856, 1994
Losman et al, Int. J. Cancer 46:310, 1990
Madzak et al, J Gen Virol 73:1533-1536, 1992
Maeda et al, J. Biol. Chem. 276(5):3628-3634, 2001
Mag et al, Nucleic Acids Res. 19:1437, 1991
Maniatis et al, Molecular Cloning, Cold Spring Harbor Laboratory, New York, 1989
Mann and Baltimore, J Virol 54:401-407, 1985
Margolskee, Curr Top Microbiol Immunol 158:67-95, 1992
Mayne et al, Eur J Immunol. 34:1217-1227, 2004
Mesmaeker et al, Bioorganic & Medicinal Chem. Lett. 4:395, 1994
Metal Radionuclide Labeled Proteins for Diagnosis and Therapy U.S. Pat. No. 4,897,255
Metal Radionuclide Chelating Compounds for Improved Chelation Kinetics U.S. Pat. No. 4,988,496
Midorikawa et al, Int. J. Cancer 103(4):455-465, 2003
Miller et al, J Virol 62:4337-4345, 1988
Miller et al, Mol Cell Biol 5:431-437, 1985
Miller, Curr Top Microbiol Immunol 158:1-24, 1992
Morrison et al, Proc. Nat. Acad. Sci. USA, 81:6851-6855, 1984
Moss, Curr Top Microbiol Immunol 158: 5-38, 1992
Moss, Proc Natl Acad Sci USA 93:11341-11348, 1996
Mountain A and Adair J R in Biotechnology and Genetic Engineering Reviews (ed. Tombs, M. 10, Chapter 1) Intercept, Andover, UK, 1992
Muenke and Wilkie Craniosynostosis Syndromes 3:6117-6146, 2000
Muzyczka, Curr Top Microbiol Immunol 158:97-129, 1992
Naldini et al, Science 272:263-267, 1996
Nisonoff et al, Arch Biochem. Biophys. 89:230, 1960
Ohi et al, Gene 89:279-282, 1990
Page et al, J Virol 64:5270-5276, 1990
Paine-Saunders et al, Dev. Biol. 225(1):179-187, 2000
Pastinen, Genome Res. 7:606-6145, 1997
Patton, Nat Biotech 16:141-143, 1998
Pauwels et al, Chemica Scripta 26:1419, 1986
Persson et al, Journal of Anatomy 125:313-321, 1978
Petropoulos et al, J Virol 66:3391-3397, 1992
Picksley et al, DNA Cloning 2: Expression Systems, 2^{nd} Edition, Glover et al (eds), page 93 Oxford University Press, 1995
Pinkel et al, Nature Genetics 20:207-211, 1998
Porter, Biochem J. 73:119, 1959
Putney and Burke, Nat Biotech 16:153-157, 1998
Quantin et al, Proc Natl Acad Sci USA 89:2581-2584, 1992
Rawls, C & E News:35, 1997
Remington's Pharmaceutical Sciences, 18^{th} Edition, Mack Publishing Company, Easton, Pa., 1990
Robbins Robbins Pathologic Basis of Disease, 1999
Rosenfeld et al, Cell 68:143-155, 1992
Rudiger et al, Proc. Natl. Acad. Sci. USA 95(5):2406-2411, 1998
Russell and Hirata, Nat Genetics 18:323-328, 1998
Samanen et al, J Pharm Pharmacol 48:119-135, 1996
Sambrook et al, Molecular Cloning: A Laboratory Manual (2^{nd} ed.) 1-3:1989
Sanger et al, Proc Natl. Acad Sci USA 74:5463, 1977
Sawai et al, Chem. Lett. 805:1984
Sayani and Chien, Crit. Rev Ther Drug Carrier Syst 13:85-184, 1996
Schneider et al, Nat Genetics 18:180-183, 1998
Scope, Protein Purification, Springer-Verlag, NY, 1982
Shapiro, “Practical flow cytometry”, 3^{rd} ed. Brisbane, Wiley-Liss, 1995
Shimada et al, J Clin Invest 88:1043-1047, 1991
Song et al, J. Cell Physiol. 202(1):255-262, 2005
Song et al, J. Biol. Chem. 280(3):2116-2125, 2005
Sorge et al, Mol Cell Biol 4:1730-1737, 1984
Sprinzl et al, Eur. J. Biochem. 81:579, 1977
Stratford-Perricaudet et al, Hum Gene Ther 1:241-256, 1990
Suntres and Shek, J Pharm Pharmacol 46:23-28, 1994
Szoka and Papahadjopoulos, Ann Rev Biophys Bioeng 9:467-508, 1980
Taylor et al, Int. Immun. 6:579, 1994
Tetrahedron, Lett. 37:743, 1996
Tijssen ed, Chapter 3 of Laboratory Techniques in Biochemistry and Molecular Biology: Hybridization with Nucleic Acid Probes, Part I, Theory and Nucleic Acid Preparation, Elsevier, N.Y. and Tijssen ed.
U.S. Pat. No. 4,744,981
U.S. Pat. No. 4,016,043
U.S. Pat. No. 4,018,653
U.S. Pat. No. 4,235,871
U.S. Pat. No. 4,424,279
U.S. Pat. No. 4,469,863
U.S. Pat. No. 4,501,728
U.S. Pat. No. 4,837,028
U.S. Pat. No. 5,034,506
U.S. Pat. No. 5,143,854
U.S. Pat. No. 5,216,141
U.S. Pat. No. 5,235,033
U.S. Pat. No. 5,279,833
U.S. Pat. No. 5,283,185
U.S. Pat. No. 5,386,023
U.S. Pat. No. 5,391,377
U.S. Pat. No. 5,445,934
U.S. Pat. No. 5,602,240
U.S. Pat. No. 5,637,684
U.S. Pat. No. 5,644,048
U.S. Pat. No. 5,744,305
U.S. Pat. No. 5,800,992
U.S. Pat. No. 5,807,522
U.S. Pat. No. 6,096,716
U.S. Pat. No. 6,110,490
Vaes et al, Bone 36(5):803-811, 2005
Vutla et al, J Pharm Sci 85:5-8, 1996
Ward et al, Monoclonal Antibodies: Principles and Applications Birch et al, Wiley-Liss, Inc.:137, 1995
Wilchek and Bayer, Anal. Biochem. 171:1-32, 1988
Wilkie Am J Med Genet. 90(1):82-84, 2000
Wilkie and Morriss-Kay Nat Rev Genet. 2(6):458-468, 2001
Wilkinson et al, Nucleic Acids Res 20:233-2239, 1992
Williams et al, DNA Cloning 2: Expression Systems, 2^{nd} Ed., Oxford University Press 1995
WO 90/15070
WO 91/09967
WO 92/10092
WO 96/17958
Woodle et al, Pharm Res 9:260-265, 1992
Wu and Wallace, Genomics 4:560, 1989
Yip et al, Teratology 21(1):29-38, 1980
Zalipsky et al, Bioconjug Chem 6:705-708, 1995
Zhao et al, Hum. Mol. Genet. 4(4):589-597, 1995

1. A method for the treatment of a bone pathology or reducing the risk of development of a bone pathology in a subject, said method comprising administering to said subject an effective amount of an agent which modulates expression of a gene or the activity of a product encoded by the gene, said gene selected for the group consisting of GPC3, RBP4, C1 QTNF3 and ANXA3 or a mammalian homolog thereof.

2. A method for the treatment of a suture-based cranial disorder, bone cancer, skeletal disorder or bone injury selected from the group consisting of a fracture, greenstick and bone chip in a subject said method comprising administering to said subject an effective amount of an agent which modulates expression of a gene or the activity of a product encoded by the gene, said gene selected for the group consisting of WIF1 and SHOX2 or a mammalian homolog thereof.

3. The method of claim 1 or 2 wherein the subject is a human.

4. The method of claim 1 or 2 wherein the bone pathology is selected from the group consisting of bone cancer, a deficient bone mineralization condition, bone injury, suture-based cranial disorder, skeletal disorder and osteoporosis.

5. The method of claim 4 wherein the bone injury is selected from the group consisting of a fracture, green stick and bone chip.

6. The method of claim 2 wherein the suture-based cranial disorder is craniosynostosis.

7. The method of claim 4 wherein the deficient bone mineralization condition is a dysplasia.

8. The method of claim 1 wherein the gene is GPC3.

9. The method of claim 1 wherein the gene is RBP4.

10. The method of claim 1 wherein the gene is C1QTNF3.

11. The method of claim 1 wherein the gene is ANXA3.

12. The method of claim 2 wherein the gene is WIF1.

13. The method of claim 2 wherein the gene is SHOX2.

14. A method for treating a subject to improve bone growth, inhibit bone growth and/or inhibit bone cancer growth in said subject said method comprising administering to said subject an effective amount of an agent which modulates expression of a gene or the activity of a product encoded by the gene, said gene selected from the group consisting of GPC3, RBP4, C1QTNF3 and ANXA3 or a mammalian homolog thereof.

15. A method for treating craniosynostosis in a subject said method comprising administering to said subject an effective amount of an agent which modulates expression of a gene or the activity of a product encoded by the gene, said gene selected for the group consisting of WIF1 and SHOX2 or a mammalian homolog thereof.

16. The method of claim 14 or 15 wherein the subject is a human.

17. The method of claim 14 wherein the gene is GPC3.

18. The method of claim 14 wherein the gene is RBP4.

19. The method of claim 14 wherein the gene is C1QTNF3.

20. The method of claim 14 wherein the gene is ANXA3.

21. The method of claim 15 wherein the gene is WIF1.

22. The method of claim 15 wherein the gene is SHOX2.

23. A method for diagnosis or prognosing a bone pathology in a subject said method comprising determining the expression profile of one or more genes selected from the group consisting of GPC3, RBP4, C1QTNF3, ANXA3, WIF1 and SHOX2 wherein an elevation in expression of GPC3, RBP4 and C1QTNF3 in unfused tissues and elevation of expression of ANXA3, WIF1 and SHOX2 in fused or fusing sutures is indicative of the presence of a bone pathology or a predisposition to development of a bone pathology.

24. The method of claim 23 wherein the subject is a human.

25. The method of claim 23 wherein the bone pathology is selected from the group consisting of bone cancer, a deficient bone mineralization condition, bone injury, suture-based cranial disorder, skeletal disorder and osteoporosis.

26. The method of claim 25 wherein the bone injury is selected from the group consisting of a fracture, green stick and bone chip.

27. The method of claim 25 wherein the suture-based cranial disorder is craniosynostosis.

28. The method of claim 25 wherein the deficient bone mineralization condition is a dysplasia.

29. A pharmaceutical composition when used in the method of treatment of claim 1 or 2 or 14 or 15 comprising an agent which modulates expression of a gene or expression product of a gene selected from the group consisting of GPC3, RBP4, C1QTNF3, ANXA3, WIF1 and SHOX2 and one or more pharmaceutically acceptable carriers, diluents and/or excipients.

30. An isolated agent which modulates expression of a gene selected from the group consisting of GPC3, RBP4, C1QTNF3, ANXA3, WIF1 and SHOX2 or the activity of the expression product from GPC3, RBP4, C1QTNF3, ANXA3, WIF1 or SHOX2 when used in the method of treatment of claim 1 or 2 or 14 or 15.

31. The method of claim 4 wherein the suture-based cranial disorder is craniosynostosis.
